(19) United States
(12) Patent Application Publication
(10) Pub. No.: US 2026/0199371 A1
(43) Pub. Date: Jul. 16, 2026

(54) COORDINATION COMPOUND, COMPOSITIONS, AND USES THEREOF FOR TREATING CANCERS

(71) Applicant: HUEIYUAN BIOTECH LLC, New York, NY (US)

(72) Inventors: HSUEH-FA PIEN, New Taipei City (TW); CHANG-YUAN TSENG, Yilan County (TW); CHIA-HSUAN PIEN, New Taipei City (TW); JENN-LINE SHEU, Hsinchu County (TW)

(21) Appl. No.: 19/136,898

(22) PCT Filed: Nov. 29, 2023
(86) PCT No.: PCT/US2023/081481

§ 371 Date: Jun. 9, 2025

(51) Int. Cl. A61K 31/555; A61K 31/475; A61K 31/513; A61K 31/704; A61K 31/7068; A61P 35/00

(52) U.S. Cl. CPC A61K 31/555; A61K 31/475; A61K 31/513; A61K 31/704; A61K 31/7068; A61P 35/00

(57) ABSTRACT

Provided is a coordination compound represented by formula (I). Also provided is a pharmaceutical composition that can be used to treat cancers, including the coordination compound of formula (I) and a pharmaceutically acceptable carrier. Also provided are applications of the coordination compound in inhibiting cancer cell survival or proliferation, inhibiting cancer cell migration or invasion, and treating cancerous tumors in a subject in need.

### CROSS-REFERENCE TO RELATED APPLICATIONS

This application claims priority of U.S. Provisional Application No. 63/431,320, filed on Dec. 9, 2022, the content of which is incorporated herein in its entirety by reference.

### SEQUENCE LISTING

The instant application contains a Sequence Listing which has been submitted electronically in WIPO Standard ST.26 format and is hereby incorporated by reference in its entirety. Said XML file, created Nov. 21, 2023, is named “PIEN-2PCT.xml” and is 10,000 bytes in size.

### FIELD

The present invention relates to a coordination compound and its use in treating neoplastic diseases. Particularly, the present invention relates to a coordination compound, a pharmaceutical composition including the coordination compound, and a method for treating cancers by using the coordination compound.

### BACKGROUND OF THE INVENTION

Cancers, a prevailing and devastating disease, continue to pose formidable challenges to medical science and healthcare systems worldwide. Despite significant advancements in our understanding of the molecular underpinnings of cancers, the quest for more effective cancer treatments persists. Current cancer treatment strategies primarily include surgery, radiation therapy, and chemotherapy. Surgery and radiation therapy are often effective only in localized, early-stage cancers, limiting their applicability to more advanced or metastatic cases. Conventional chemotherapy, involving the administration of cytotoxic drugs to tumors, has long been a cornerstone of cancer treatment. Although it has shown some success in reducing tumor size and extending patient survival, its utility is marred by significant limitations. One major limitation is that cancer cells can develop resistance to chemotherapeutic drugs, rendering them ineffective over time.

The development of drug resistance is a pervasive problem in cancer treatment. Cancer cells can evolve mechanisms to counteract the effects of chemotherapy, rendering once-effective treatments impotent and leading to patients' requirements for multiple rounds of chemotherapy with different agents. One example is colorectal cancer (CRC), a malignancy that causes significant deaths worldwide and is the third most common cause of cancer-related deaths globally. Chemotherapy with oxaliplatin (OXA) is the main treatment for advanced CRC. Though numerous studies have demonstrated its potential therapeutic effect, drug resistance remains a major challenge, making it difficult to treat CRC effectively and causing recurrent CRC.

In addition to drug resistance to chemotherapy, the presence of metastatic cancer cells also contributes to cancer recurrence, resulting in patients experiencing tumor regrowth, often in a more aggressive and treatment-resistant form. Metastasis, the spread of cancer cells from the primary tumor to distant sites in the body, is a major contributor to cancer-related mortality. This complex process involves the invasion of cancer cells into surrounding tissues, intravasation into blood or lymphatic vessels, circulation through the bloodstream, extravasation at secondary sites, and subsequent growth into secondary tumors. Metastatic cancers are often highly aggressive and resistant to treatment, presenting a formidable challenge for clinicians.

In light of the above challenges, there is an urgent and unmet need for innovative solutions in treating cancers, particularly in preventing cancer progression.

### SUMMARY OF THE INVENTION

The present disclosure concerns a coordination compound that can be used as an anticancer agent to inhibit the survival, proliferation, migration, or invasion of cancer cells. The coordination compound is represented by formula (I): [XZ₂(CH₃CO₂)₆(H₂O)₄(OH)₂]NO₃. X and Z in formula (I) refer to different metal ions, wherein X is a trivalent ion of chromium (Cr) or molybdenum (Mo), and Z is a trivalent ion of iron (Fe), ruthenium (Ru), or osmium (Os).

In one aspect, the present disclosure relates to a method for inhibiting survival or proliferation of a cancer cell, including contacting the cancer cell with an effective amount of the coordination compound represented by formula (I), wherein X is a trivalent ion of chromium or molybdenum, and Z is a trivalent ion of iron, ruthenium, or osmium. Preferably, the coordination compound includes a chromium(III) ion and an iron(II) ion and is represented by formula (II).

The cytotoxic effect of the coordination compound applies to a broad spectrum of cancer cells. In some embodiments, the cancer is selected from colorectal cancer, lung cancer, liver cancer, pancreatic cancer, bone cancer (including osteosarcoma), brain cancer (including glioblastoma), breast cancer, ovarian cancer, cervical cancer, prostate cancer, bladder cancer (including transitional cell carcinoma), blood cancer (including leukemia and lymphoma), gastric cancer, skin cancer (including melanoma), head-and-neck cancer (including oral squamous cell carcinoma), or thyroid cancer. In some embodiments, the cancer cell is an invasive cancer cell. In some embodiments, the cancer cell is resistant to a chemotherapeutic agent such as oxaliplatin.

In some embodiments, the coordination compound induces endoplasmic reticulum (ER) stress-mediated apoptosis of the cancer cell, thereby inhibiting the survival or proliferation of the cancer cell.

In some embodiments, the method for inhibiting cancer cell survival or proliferation further includes the step of contacting the cancer cell with a chemotherapeutic agent selected from oxaliplatin, irinotecan, 5-fluorouracil, gemcitabine, doxorubicin, or any combination thereof.

In another aspect, the present disclosure relates to a method for inhibiting migration or invasion of a cancer cell, including contacting the cancer cell with an effective amount of the coordination compound represented by formula (I), wherein X is a trivalent ion of chromium or molybdenum, and Z is a trivalent ion of iron, ruthenium, or osmium. Preferably, the coordination compound includes a chromium(II) ion and an iron(II) ion and is represented by formula (II).

The migration or invasion inhibiting effect of the coordination compound applies to multiple types of cancer cells. In some embodiments, the cancer is colorectal cancer or bone cancer. In some embodiments, the cancer cell is an invasive cancer cell. In some embodiments, the cancer cell is resistant to a chemotherapeutic agent such as oxaliplatin.

In some embodiments, the coordination compound suppresses epithelial-mesenchymal transition (EMT) of the cancer cell, thereby inhibiting the migration or invasion of the cancer cell.

In still another aspect, the present disclosure relates to a method for or treating a cancerous tumor, including administering to a subject in need an effective amount of the coordination compound represented by formula (I), wherein X is a trivalent ion of chromium or molybdenum, and Z is a trivalent ion of iron, ruthenium, or osmium. Preferably, the coordination compound includes a chromium(II) ion and an iron(II) ion and is represented by formula (II).

In some embodiments, the cancerous tumor is selected from colorectal cancer, lung cancer, liver cancer, pancreatic cancer, bone cancer, brain cancer, breast cancer, ovarian cancer, cervical cancer, prostate cancer, bladder cancer, blood cancer, gastric cancer, skin cancer, head-and-neck cancer, or thyroid cancer. In some embodiments, the cancerous tumor includes an invasive cancer cell. In some embodiments, the cancerous tumor includes a cancer cell resistant to a chemotherapeutic agent such as oxaliplatin.

The present disclosure further relates to a pharmaceutical composition, which at least can be used to treat cancers. The pharmaceutical composition includes an effective amount of the coordination compound represented by formula (I) and a pharmaceutically acceptable carrier, wherein X is a trivalent ion of chromium or molybdenum, and Z is a trivalent ion of iron, ruthenium, or osmium. Preferably, the coordination compound includes a chromium(II) ion and an iron(III) ion and is represented by formula (II).

In some embodiments, the pharmaceutical composition further includes an additional pharmaceutically active agent such as a chemotherapeutic agent or an immunomodulator. In some embodiments, the additional pharmaceutically active agent is a chemotherapeutic agent selected from oxaliplatin, irinotecan, 5-fluorouracil, gemcitabine, doxorubicin, or any combination thereof.

The coordination compound disclosed herein can target multiple factors associated with cancer progression and metastasis and is effective against chemo-resistant cancer cells. Therefore, the compound can be used in medical preparations for treating cancers, including but not limited to oxaliplatin-resistant colorectal cancer.

### BRIEF DESCRIPTION OF DRAWINGS

The present invention will be apparent to those skilled in the art from the following detailed description of the preferred embodiments, with reference to the accompanying drawings, in which:

FIG. 1A is the ¹H-NMR spectrum of the coordination compound of formula (II);

FIG. 1B is the ¹³C-NMR spectrum of the coordination compound of formula (II);

FIG. 1C is the FT-IR spectrum of the coordination compound of formula (II);

FIG. 2A is a schematic diagram illustrating the process of establishing oxaliplatin-resistant LoVo cells (referred to as LoVo-OXAR cells) from LoVo-parental cells;

FIG. 2B shows the light micrographs of LoVo-parental cells and LoVo-OXAR cells;

FIG. 2C shows the cytotoxic effects of oxaliplatin on LoVo-parental cells and LoVo-OXAR cells; * and ** indicate p<0.05 and p<0.01, respectively, compared to no oxaliplatin treatment (control group);

FIG. 2D shows the effect of the coordination compound of formula (II) on the viability of LoVo-parental cells; *, ** and *** indicate p<0.05, p<0.01, and p<0.001, respectively, compared to no compound treatment (control group);

FIG. 2E shows the effect of the coordination compound of formula (II) on the viability of LoVo-OXAR cells; * and ** indicate p<0.05 and p<0.01, respectively, compared to no compound treatment (control group);

FIG. 2F shows the synergistic cytotoxic effects of the coordination compound of formula (II) in combination with oxaliplatin (OXA), irinotecan, or 5-fluorouracil (5-FU) on LoVo-parental cells and LoVo-OXAR cells; * and *** indicate p<0.05 and p<0.001, respectively, compared to no compound treatment to LoVo-parental cells (control group); ^{#} and ^{###} indicate p<0.05 and p<0.001, respectively, compared to no compound treatment to LoVo-OXAR cells (control group);

FIG. 3A shows the effect of the coordination compound of formula (II) on the viability of CL1-parental cells;

FIG. 3B shows the effect of the coordination compound of formula (II) on the viability of gemcitabine-resistant CL1 cells (referred to as CL1-GEMR cells);

FIG. 3C shows the effect of the coordination compound of formula (II) on the viability of BEAS-2B cells;

FIG. 4A shows the effect of the coordination compound of formula (II) on apoptosis in LoVo-parental cells; ** indicates p<0.01 compared to no compound treatment (control group);

FIG. 4B shows the effect of the coordination compound of formula (II) on apoptosis in LoVo-OXAR cells; ** indicates p<0.01 compared to no compound treatment (control group);

FIG. 4C shows western blot analysis of ER stress markers in LoVo-parental and LoVo-OXAR cells in the presence or absence of the coordination compound of formula (II) or oxaliplatin (OXA);

FIG. 5A shows the effect of the coordination compound of formula (II) on the migration of LoVo-parental and LoVo-OXAR cells; *, ** and *** indicate p<0.05, p<0.01, and p<0.001, respectively, compared to no compound treatment to LoVo-parental cells (control group); , ^{#}, ^{##}, and ^{###} indicate p<0.05, p<0.01, and p<0.001, respectively, compared to no compound treatment to LoVo-OXAR cells (control group);

FIG. 5B shows the effect of the coordination compound of formula (II) on the invasion of LoVo-parental and LoVo-OXAR cells; ** and *** indicate p<0.01, and p<0.001, respectively, compared to no compound treatment to LoVo-parental cells (control group); ^{#} and ^{##} indicate p<0.05 and p<0.01, respectively, compared to no compound treatment to LoVo-OXAR cells (control group);

FIG. 5C shows western blot analysis of the EMT markers in LoVo-parental cells treated with or without the coordination compound of formula (II);

FIG. 5D shows western blot analysis of the EMT markers in LoVo-OXAR cells treated with or without the coordination compound of formula (II);

FIG. 5E shows the effect of the coordination compound of formula (II) on the EMT marker expression in LoVo-parental cells, which were assayed by immunofluorescence staining; ** indicates p<0.01 compared to no compound treatment (control group);

FIG. 5F shows the effect of the coordination compound of formula (II) on the EMT marker expression in LoVo-OXAR cells, which were assayed by immunofluorescence staining; * and ** indicate p<0.05 and p<0.01, respectively, compared to no compound treatment (control group);

FIG. 5G shows the effect of the coordination compound of formula (II) on the mRNA expression of EMT markers in LoVo-parental cells; *, ** and *** indicate p<0.05, p<0.01, and p<0.001, respectively, compared to no compound treatment (control group);

FIG. 5H shows the effect of the coordination compound of formula (II) on the mRNA expression of EMT markers in LoVo-OXAR cells; *, ** and *** indicate p<0.05, p<0.01, and p<0.001, respectively, compared to no compound treatment (control group);

FIG. 6A shows representative pictures of tumor masses isolated from mice of different treatment groups on day 21 after tumor cell inoculation;

FIG. 6B shows the tumor growth in mice of different treatment groups from day 1 to day 21 after tumor cell inoculation;

FIG. 6C shows the body weight of mice of different treatment groups from day 1 to day 21 after tumor cell inoculation;

FIG. 6D shows the effect of the coordination compound of formula (II) on apoptosis in tumors of mice inoculated with LoVo-parental cells; ** indicates p<0.01 compared to no compound treatment (control group);

FIG. 6E shows the effect of the coordination compound of formula (II) on apoptosis in tumors of mice inoculated with LoVo-OXAR cells; ** indicates p<0.01 compared to no compound treatment (control group);

FIG. 6F shows representative pictures of mouse organs demonstrating the effect of the coordination compound of formula (II) on LoVo-parental tumor metastasis in mice of different treatment groups; arrows indicate metastatic cancer cell growth;

FIG. 6G shows representative pictures of mouse organs demonstrating the effect of the coordination compound of formula (II) on LoVo-OXAR tumor metastasis in mice of different treatment groups; arrows indicate metastatic cancer cell growth;

FIG. 7 shows the effect of the coordination compound of formula (II) (denoted as Com. (II)) on the viability of HA22T-patental cells and SAHA-resistant HA22T cells (referred to as HA22T-HDACiR cells); *, **, and *** indicate p<0.05, p<0.01, and p<0.001, respectively, compared to no compound treatment (control group);

FIG. 8 shows the effect of the coordination compound of formula (II) on the viability of LNCaP cells;

FIG. 9A shows the effect of the coordination compound of formula (II) on the viability of 143B cells;

FIG. 9B shows the synergistic cytotoxic effects of the coordination compound of formula (II) in combination with 5-fluorouracil (5-FU), gemcitabine, or doxorubicin on 143B cells;

FIG. 9C shows the effect of the coordination compound of formula (II) on the migration of 143B cells;

FIG. 9D shows western blot analysis of the EMT markers in 143B cells treated with or without the coordination compound of formula (II);

FIG. 10 shows the effect of the coordination compound of formula (II) on the viability of GBM 8401 cells;

FIG. 11 shows the effect of the coordination compound of formula (II) (denoted as Com. (II)) on the viability of T-47D and MDA-MB231 cells;

FIG. 12 shows the effect of the coordination compound of formula (II) on the viability of T24 cells;

FIG. 13 shows the effect of the coordination compound of formula (II) on the viability of SCC-25 cells;

FIG. 14 shows the effect of the coordination compound of formula (II) on the viability of CP70 cells;

FIG. 15 shows the effect of the coordination compound of formula (II) on the viability of Jurkat cells;

FIG. 16 shows the effect of the coordination compound of formula (II) on the viability of HeLa cells;

FIG. 17 shows the effect of the coordination compound of formula (II) on the viability of TT cells;

FIG. 18 shows the effect of the coordination compound of formula (II) on the viability of A431 cells;

FIG. 19 shows the effect of the coordination compound of formula (II) on the viability of MIA PaCa-2 and PANC-1 cells;

FIG. 20A shows the tumor growth in mice of different treatment groups after tumor cell inoculation; and

FIG. 20B shows the body weight of mice of different treatment groups after tumor cell inoculation.

### DETAILED DESCRIPTION OF THE INVENTION

The present invention is further explained in the following embodiments and examples. It is understood that the examples given below do not limit the scope of the invention, and it will be evident to those skilled in the art that modifications can be made without departing from the scope of the appended claims.

Unless defined otherwise, all technical and scientific terms and abbreviations used herein have the same meaning as commonly understood by a person skilled in the art to which this invention pertains.

### Definition

As used herein, the singular forms “a”, “an” and “the” include plural referents unless the context clearly indicates otherwise. For example, reference to “a pharmaceutically acceptable carrier” includes a mixture of pharmaceutically acceptable carriers, and reference to “an additional pharmaceutically active agent” includes more than one pharmaceutically active agent.

Data are generally presented as mean±standard deviation. Numerical quantities given herein are approximate, and experimental values may vary within 20 percent, preferably within 10 percent, or most preferably within 5 percent. Thus, the terms “about” and “approximately” refer to within 20 percent, preferably within 10 percent, or most preferably within 5 percent of a given value or range.

The term “tumor(s),” as used herein, means an aberrant cell mass characterized by unregulated cell proliferation and growth. Tumors encompass solid tumors, which form distinct masses, and non-solid tumors, such as leukemia, which involve abnormal cell proliferation in the blood or other bodily fluids. Tumors may be noncancerous (also called benign) or cancerous (also called malignant) tumors. Noncancerous tumors contain relatively slow-growing tumor cells whose expansion is confined to their site of origin and thus do not spread to other parts of a subject's body. Cancerous tumors (used interchangeably with the term “cancers”) contain quickly dividing tumor cells that have the potential to invade nearby tissues or spread to distant organs when present in a subject's body. Based on the context of the disclosure, the term “tumor” may specifically refer to “cancerous tumor,” and the term “tumor cell” may specifically refer to “cancer cell.”

The term “cancer cell,” as used herein, may refer to a single cancer cell or a population of homogenous or heterogeneous cancer cells. Cancer cells may be those in a subject's body, isolated from a subject (such as a human), or derivative cells of isolated cancer cells. Also, cancer cells may have a variety of sources unless otherwise specified.

The term “coordination compound,” as used herein, refers to a metal complex defined by formula (I), which includes three metal ion centers each surrounded by six ligands.

### Coordination Compound

The coordination compound disclosed herein is represented by formula (I): [XZ₂(CH₃CO₂)₆(H₂O)₄(OH)₂]NO₃. X may be a trivalent ion of chromium (Cr) or molybdenum (Mo), and Z may be a trivalent ion of iron (Fe), ruthenium (Ru), or osmium (Os). Since the coordination compound may contain Cr or Mo, both being a group 6 metal, as well as Fe, Ru, or Os, all being a group 8 metal, the coordination compound may be represented by formula (II), (III), (IV), (V), (VI), or (VII), depending on the various combinations of central metal ions:

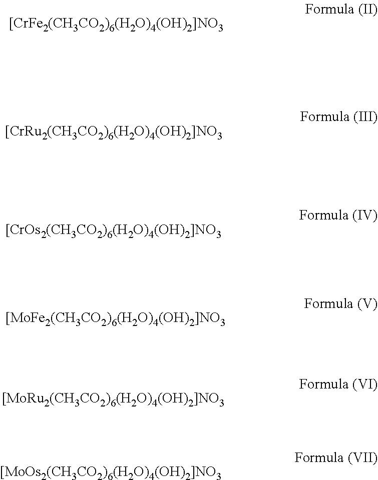

In some embodiments, the coordination compound includes a chromium(II) ion and an iron(II) ion and is represented by formula (II). The compound of formula (II) may have the structure (a) shown below or other structures representing different stereoisomers. That is, the compound of formula (II) refers to any compounds with formula (II) that may have different arrangement of ligands around the metal ions. Likewise, the compound of formula (III), (IV), (V), (VI), or (VII) may include various stereoisomers.

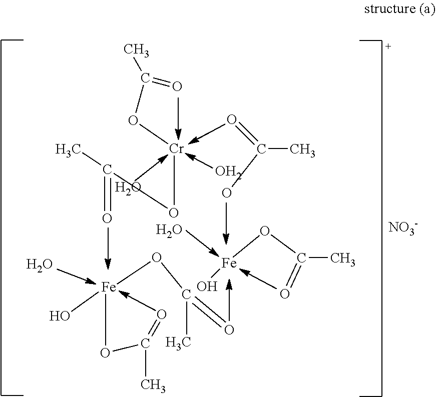

### Use of the Coordination Compound in Inhibiting Cancer Cell Survival or Proliferation

The present disclosure provides a method for inhibiting survival or proliferation of a cancer cell, including contacting the cancer cell with an effective amount of the coordination compound represented by formula (I): [XZ₂(CH₃CO₂)₆(H₂O)₄(OH)₂]NO₃, wherein X is a trivalent ion of chromium or molybdenum, and Z is a trivalent ion of iron, ruthenium, or osmium. The term “inhibiting survival or proliferation,” as used herein, refers to preventing cancer cells from continuing to exist or slowing down or stopping cancer cells from expanding after the use of the coordination compound. Said inhibition may be assessed by methods known in the art, for example, direct cell counting, monitoring cell proliferation over time with a real-time cell analysis system, determining cell viability by measuring metabolically active cells (such as MTT assay), measuring DNA synthesis to assess cell division, and measuring the size of a tumor.

The cytotoxicity of the coordination compound has been demonstrated in various cancer cells, including colorectal cancer cells, lung cancer cells, liver cancer cells, pancreatic cancer cells, bone cancer cells, brain cancer cells, breast cancer cells, ovarian cancer cells, cervical cancer cells, prostate cancer cells, bladder cancer cells, blood cancer cells, gastric cancer cells, skin cancer cells, head-and-neck cancer cells, and thyroid cancer cells. In some embodiments, the coordination compound exerts the cytotoxic effect on invasive cancer cells, such as invasive colorectal cancer cells. In some embodiments, the coordination compound exerts the cytotoxic effect on cancer cells that are resistant to a chemotherapeutic agent, such as oxaliplatin-resistant colorectal cancer cells.

The effective amount of the coordination compound applied to a cancer cell to inhibit cell survival or proliferation refers to the amount sufficient to cause cancer cell death or limiting the proliferation of cancer cells. Effective amounts will vary, as recognized by those skilled in the art, depending on factors such as the type of cancer cells, the specific coordination compound being applied, how the coordination compound is delivered to cancer cells, and the co-usage of the coordination compound and other anti-tumor agents.

The step of contacting a cancer cell with the coordination compound may be carried out by methods known in the art. In some embodiments, a cancer cell is in contact with the coordination compound by being incubated in a cell culture medium supplemented with the coordination compound. In some embodiments, a cancer cell is in contact with the coordination compound by being exposed, in vitro or in vivo, to a composition containing the coordination compound and a pharmaceutically acceptable carrier.

In some embodiments, the coordination compound inhibits cancer cell survival or proliferation by inducing endoplasmic reticulum (ER) stress-mediated apoptosis of cancer cells. For example, the coordination compound of formula (II) induces apoptosis of colorectal cancer cells and thus inhibits their survival, reducing cell number and blocking proliferation. Such apoptosis-inducing effect may result from activated and sustained ER stress in cancer cells, which accompanies unresolvable unfolded protein response (UPR) and triggers cancer cell death. The induced apoptosis inhibits the growth of existing cancer cells (including chemo-resistant cancer cells) and can also prevent the development of chemo-resistant cancer cell populations.

In some embodiments, the method for inhibiting cancer cell survival or proliferation further includes the step of contacting the cancer cell with a chemotherapeutic agent selected from oxaliplatin, irinotecan, 5-fluorouracil, gemcitabine, doxorubicin, or any combination thereof. In other words, the coordination compound may be employed in combination therapy with certain chemotherapeutic drugs. The coordination compound and other chemotherapeutic drugs may be co-administered to cells or a subject in need concurrently or sequentially.

### Use of the Coordination Compound in Inhibiting Cancer Cell Migration or Invasion

The present disclosure provides a method for inhibiting migration or invasion of a cancer cell, including contacting the cancer cell with an effective amount of the coordination compound represented by formula (I): [XZ₂(CH₃CO₂)₆(H₂O)₄(OH)₂]NO₃, wherein X is a trivalent ion of chromium or molybdenum, and Z is a trivalent ion of iron, ruthenium, or osmium. The term “inhibiting migration or invasion,” as used herein, refers to preventing the acquisition of or reducing the ability of a cancer cell to move from its original location or to penetrate and spread into surrounding or distal environments after the use of the coordination compound. Said inhibition may be assessed by methods known in the art, for example, live cell imaging with microscopy, measuring the movement of cells across a porous membrane in a chamber system (such as Transwell migration or invasion assay), assessing changes in expression of essential genes or proteins involved in cell migration and invasion pathways using molecular techniques like quantitative polymerase chain reaction (qPCR) and western blotting, and monitoring metastasis of cancerous tumors in a subject.

In some embodiments, the coordination compound can inhibit the migration or invasion of colorectal cancer cells or bone cancer cells. In some embodiments, the coordination compound inhibits the migration or invasion of invasive cancer cells, such as invasive colorectal cancer cells or crawling osteosarcoma cells. In some embodiments, the coordination compound is capable of inhibiting migration or invasion of cancer cells that are resistant to a chemotherapeutic agent, such as oxaliplatin-resistant colorectal cancer cells.

The effective amount of the coordination compound applied to a cancer cell to inhibit cell migration or invasion refers to the amount sufficient to prevent noninvasive cancer cells from transforming into migratory or invasive cells or reduce the capability of cancer cells to migrate or invade from a primary location to a secondary location. Effective amounts will vary, as recognized by those skilled in the art, depending on factors such as the type of cancer cells, the specific coordination compound being applied, how the coordination compound is delivered to cancer cells, and the co-usage of the coordination compound and other anti-tumor agents.

In some embodiments, the coordination compound inhibits cancer cell migration or invasion by suppressing epithelial-mesenchymal transition (EMT) of cancer cells. For example, the coordination compound of formula (II) induces the expression of epithelial phenotype markers, such as E-cadherin and tight junction protein 1 (TJP1) and represses the expression of mesenchymal phenotype markers, such as vimentin and fibronectin 1 (FN1) in colorectal cancer cells, thereby inhibiting colorectal cancer cell migration and invasion. The EMT-suppressing activity of the coordination compound may contribute to preventing both cancer metastasis and chemoresistance.

### Use of the Coordination Compound in Treating Cancers

The coordination compound has also shown therapeutic effects in subjects afflicted with cancers. Accordingly, the present disclosure further provides a method for treating a cancerous tumor, including administering to a subject in need an effective amount of the coordination compound represented by formula (I), wherein X is a trivalent ion of chromium or molybdenum, and Z is a trivalent ion of iron, ruthenium, or osmium. In some preferred embodiments, the coordination compound being administered is the compound of formula (II) described above.

The term “subject” as used herein refers to a mammal. The subject may be human or non-human, including but not limited to a primate, murine, dog, cat, bovine, goat, sheep, horse, rabbit, pig, or the like. Thus, therapeutic methods and compositions for veterinary uses and medical uses are contemplated herein.

The coordination compound may be used to treat the cancerous tumor selected from colorectal cancer, lung cancer, liver cancer, pancreatic cancer, bone cancer, brain cancer, breast cancer, ovarian cancer, cervical cancer, prostate cancer, bladder cancer, blood cancer, gastric cancer, skin cancer, head-and-neck cancer, or thyroid cancer. In some embodiments, the cancerous tumor includes a cancer cell being invasive and/or resistant to a chemotherapeutic agent such as oxaliplatin.

The effective amount of the coordination compound administered to a subject in need may be a prophylactically effective amount or a therapeutically effective amount. The “prophylactically effective amount” refers to the amount sufficient to prevent cancer progression, metastasis, the occurrence of invasive or chemo-resistant cancer cells, or the onset of symptoms or signs associated with the foregoing in a subject. The “therapeutically effective amount” refers to the amount sufficient to arrest or delay tumor growth, metastasis, the development of invasive or chemo-resistant cancer cells, or the symptoms or signs associated with the foregoing in a subject. Effective amounts will vary, as recognized by those skilled in the art, depending on factors such as the type of cancer, the age, weight, physical condition, and responsiveness of the subject to be treated, the specific coordination compound being applied, the routes of administration, excipient usage, and the co-usage with other pharmaceutically active agents.

The coordination compound of formula (I) may be administered to the subject by any suitable route, preferably in the form of a pharmaceutical composition adapted to such a route. In some embodiments, the coordination compound is administered orally, topically, transmucosally, intravenously, or intestinally. In some embodiments, the coordination compound is administered to the subject in a solid form or in a liquid form.

In some embodiments, the coordination compound of formula (I) is administered to a subject at the early stage of cancer development. In some embodiments, the coordination compound is administered to a subject at later stages of cancer development. In some embodiments, the coordination compound is administered at least once, twice, three times, or more daily. In some embodiments, the coordination compound is administered daily, every other day, weekly, several times per week, monthly, or at an even lower frequency, to maintain an effective dosage level and patient compliance. In some embodiments, the administration of the coordination compound is continued for several weeks, months, or longer. The frequency and duration of treatment may vary depending on a subject's response to treatment.

### Pharmaceutical Compositions

The present disclosure further provides a pharmaceutical composition for use in treating a cancer, including an effective amount of the coordination compound represented by formula (I) and a pharmaceutically acceptable carrier, wherein X is a trivalent ion of chromium or molybdenum and Z is a trivalent ion of iron, ruthenium, or osmium. In some preferred embodiments, the pharmaceutical composition includes the coordination compound of formula (II) as the active ingredient.

The pharmaceutical composition may be in any suitable form, such as a tablet, powder, solution, suspension, emulsion, liposomes, nanoparticles, or other preparations.

The term “pharmaceutically acceptable carrier,” as used herein, refers to any carrier or vehicle compatible with the coordination compound and other active ingredients, if any, and preferably capable of stabilizing the active ingredient and not deleterious to the subject to be treated. The pharmaceutically acceptable carrier may be excipients, diluents, antioxidants, and preservatives known in the art. Examples of pharmaceutically acceptable carriers include but are not limited to water, saline, buffers, organic solvents, hydrophilic polymers, carbohydrates, peptides, amino acids, and surfactants.

In some embodiments, the pharmaceutical composition further includes an additional pharmaceutically active agent. The term “pharmaceutically active agent” refers to a small-molecule compound or a macromolecule (such as an antibody or a fragment thereof) that possesses a desired pharmacological effect and is therapeutically effective. The pharmaceutically active agent may be a chemotherapeutic agent, an immunomodulator, or any combination thereof. Examples of chemotherapeutic agents include but are not limited to alkylating agents (e.g., cyclophosphamide, melphalan, temozolomide, carboplatin, cisplatin, and oxaliplatin), anti-metabolites (e.g., 5-fluorouracil, 6-mercaptopurine, cytarabine, gemcitabine, and methotrexate), antitumor antibiotics (e.g., actinomycin-D, bleomycin, daunorubicin, and doxorubicin), topoisomerase inhibitors (e.g., etoposide, irinotecan, teniposide, and topotecan), mitotic inhibitors (e.g., docetaxel, estramustine, nocodazole, paclitaxel, and vinblastine), histone deacetylase (HDAC) inhibitors (e.g., vorinostat, also called suberoylanilide hydroxamic acid (SAHA)), and steroids (e.g., prednisone, methylprednisolone, and dexamethasone). Examples of immunomodulators include an immunostimulant (e.g., trastuzumab) and an immunosuppressive agent.

### EXAMPLES

### Example 1: Preparation of the Coordination Compound

The procedure described below for preparing the coordination compound of formula (II) (abbreviated as “compound (II)”) is an example to illustrate the preparation of the coordination compound disclosed herein. Chromium(II) nitrate and iron(III) nitrate at a molar ratio ranging from about 1:1 to 1:3 were mixed in a flask. Alcohol with 60-95% ethanol content (the remaining being water) was poured into the flask at room temperature, where the mass-to-volume ratio of the iron(III) nitrate to the alcohol, in g/ml, was from about 1:1 to 1:3, and the mixture was stirred until all solids were dissolved. Acetic anhydride was then added into the flask such that the volume ratio of the acetic anhydride and the alcohol ranged from about 4:1 to 7:1, followed by stirring the reaction mixture for about 2-6 hours. The reaction with acetic anhydride was carried out below 70° C. Subsequently, the reaction mixture was stirred for 20-28 hours before the collection of solid precipitates of the compound (II) by filtration. The solid precipitates were dried in an oven to constant weight.

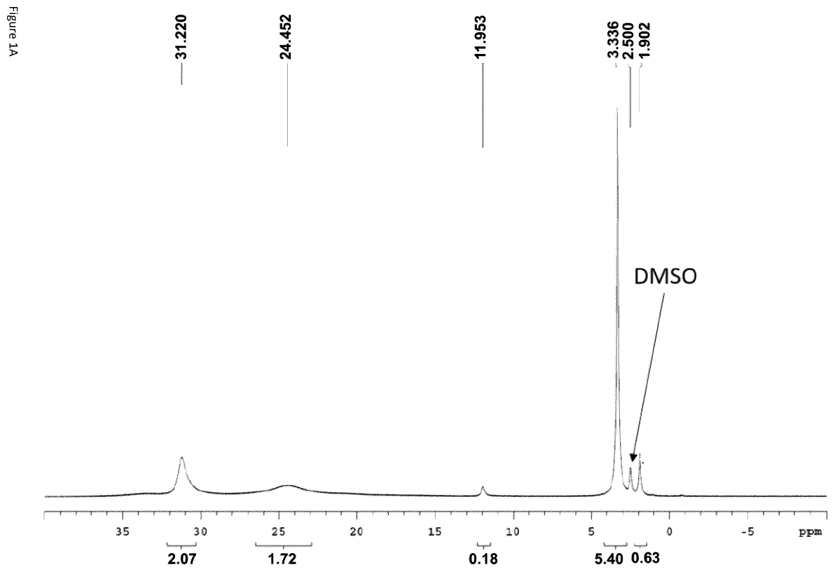

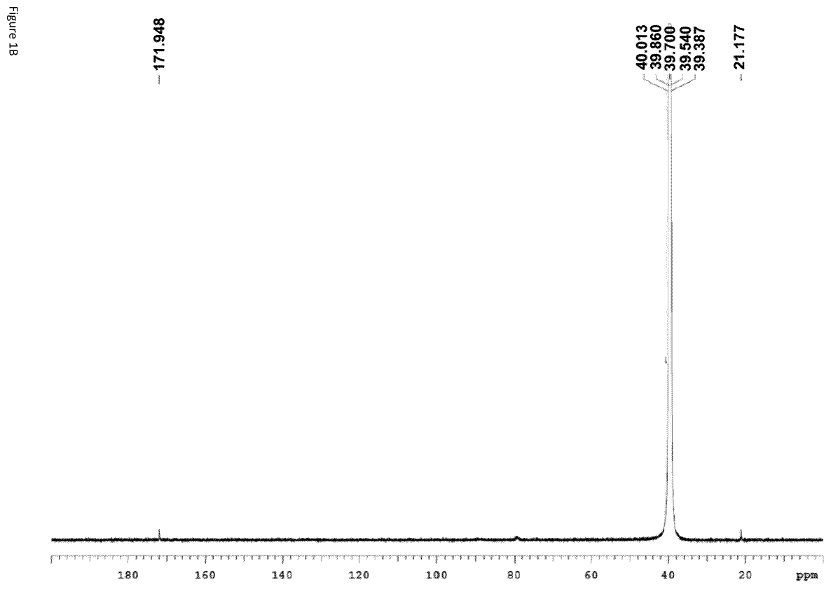

The molecular weight of the compound (II) was determined as about 686.11 by mass spectrometry. Elemental analysis showed that the compound (II) contained about 7.36% chromium, about 19.31% iron, about 2.21% nitrogen, about 21.73% carbon, about 3.86% hydrogen, and about 44.21% oxygen by mass. The structure of the compound (II) was characterized by ¹H nuclear magnetic resonance (NMR) and ¹³C-NMR spectroscopy as well as Fourier transform infrared (FT-IR) spectroscopy. FIG. 1A shows the 1H-NMR spectrum of the compound (II), where the signal at about 3.3±0.2 and 1.9±0.2 ppm corresponded to the hydrogen of H₂O and an acetate group, respectively. Also, the signals at about 21.2±0.5, 39.7±0.5, and 171.9±0.5 ppm in the ¹³C-NMR spectrum indicated the presence of an acetate group (FIG. 1B). Further, the IR spectrum showed absorption peaks at about 3400±10, 3200±10, 3000±10, 1683±10, 1585±10, 1428±10, 1349±10, 1292±10, and 1035±10 cm⁻¹ (FIG. 1C). All data supported the structure of the compound (II) as disclosed. The compound (II) was used in Examples 2-20 to study its therapeutic effects.

The coordination compounds of formula (III) to (VII) may be prepared by the process described above but with appropriate metal salts substituting chromium(II) nitrate and/or iron(II) nitrate.

### Example 2: Cytotoxic Effects of the Coordination Compound of Formula (II) on Colorectal Cancer Cells

To assess whether the coordination compound has the potential to inhibit the survival of cancer cells, including chemo-resistant cancer cells, MTT (3-(4,5-dimethylthiazol-2-yl)-2,5-diphenyltetrazolium bromide) assay was performed to measure the viability of colorectal cancer cells treated with or without the compound (II).

### 2.1 Cell Culture

Human colon cancer LoVo cells (commercially available from the American Type Culture Collection (ATCC) under the number CCL-229 or from the Bioresource Collection and Research Center (BCRC; Hsinchu, Taiwan) under the number BCRC 60148; hereinafter referred to as LoVo-parental cells) and chemo-resistant subclones thereof were cultured in Dulbecco's modified Eagle's medium (DMEM; Sigma-Aldrich, Missouri, USA) supplemented with 10% fetal bovine serum (FBS; HyClone, Utah, USA). All cell cultures were maintained at 37° C. with 5% CO₂ supply. The culture medium was replenished with fresh medium 48 hours after sub-cultivation.

### 2.2 Establishment of Oxaliplatin-Resistant Colorectal Cancer Cells

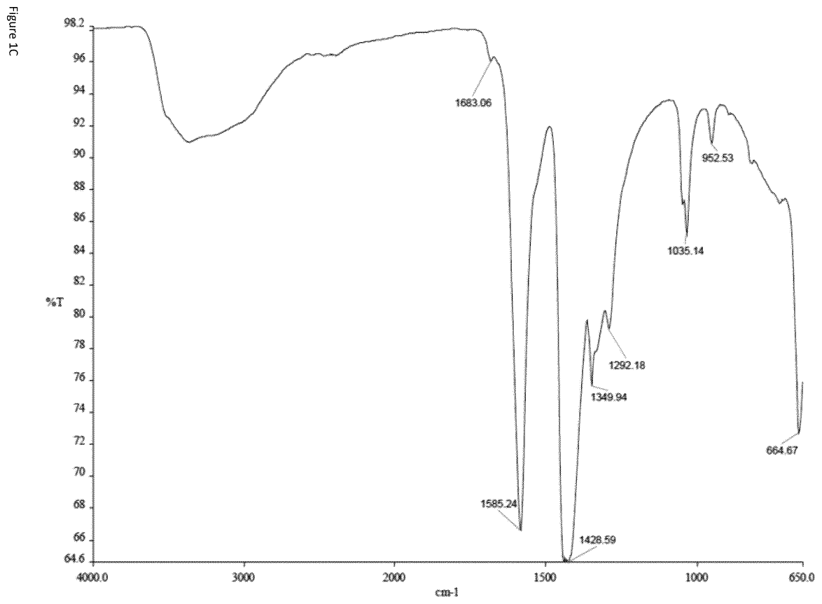

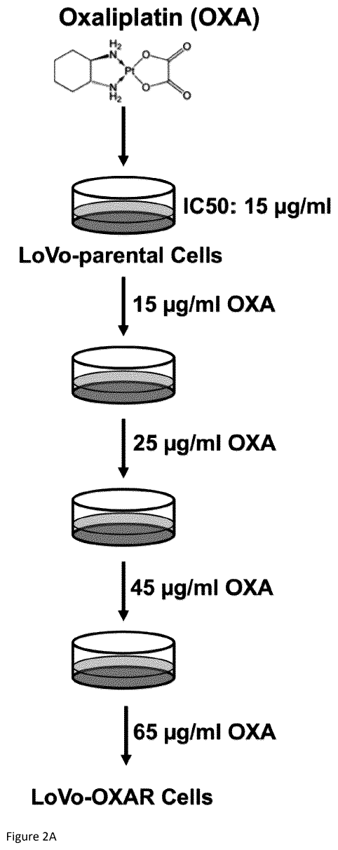

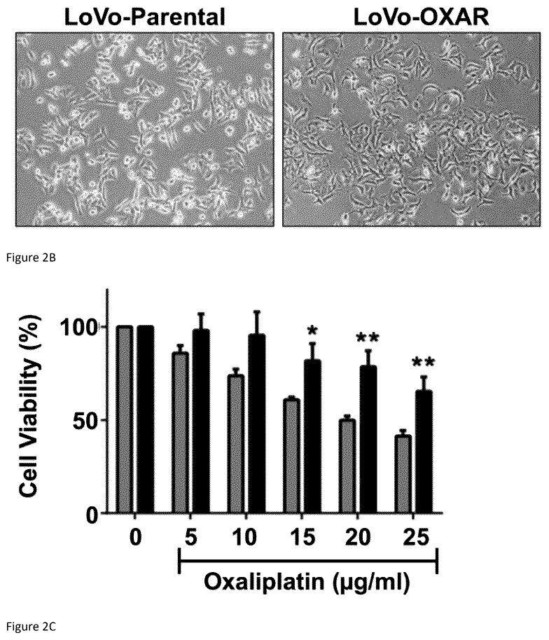

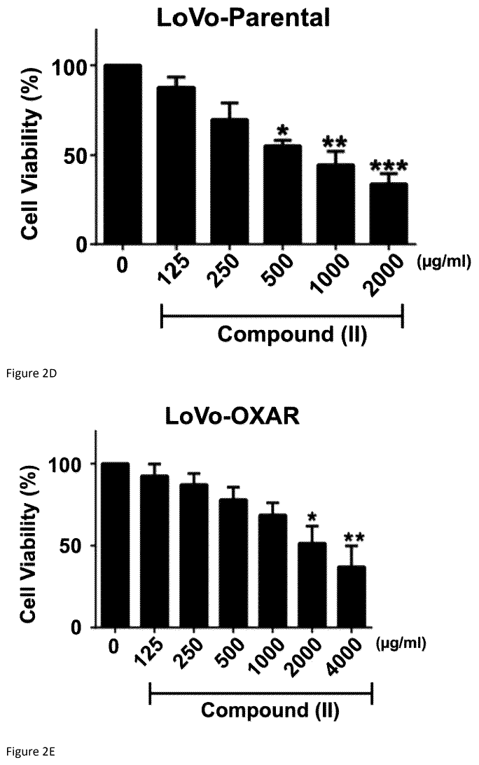

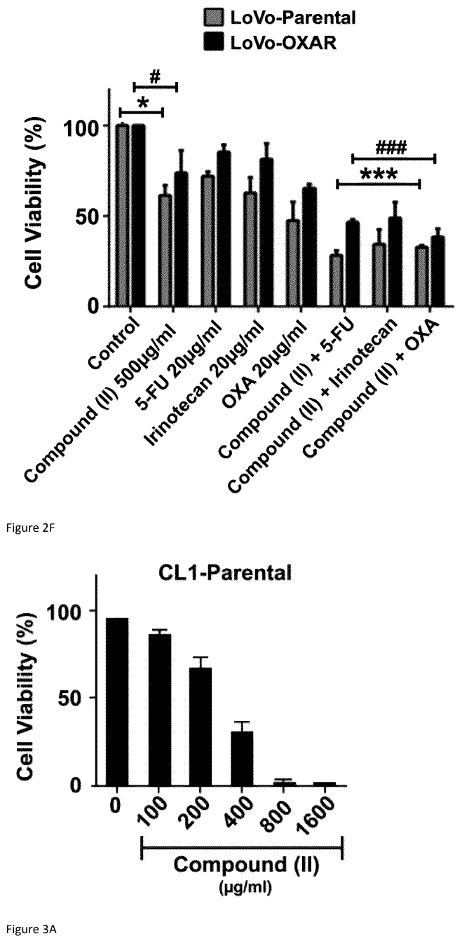

The IC50 (half maximal inhibitory concentration) of oxaliplatin (purchased from Sigma-Aldrich, #09512) in LoVo-parental cells was determined as about 15.0 μg/ml by MTT assay. For the preparation of a subclone of LoVo cells with stable resistance to oxaliplatin, the LoVo cells surviving a lower concentration (e.g., 15.0 μg/ml) of oxaliplatin treatment were exposed to a higher concentration (e.g., 25.0 μg/ml) of oxaliplatin for 24 hours, which was repeated until an oxaliplatin-resistant LoVo cell population (referred to as LoVo-OXAR cells) with an approximately 4-fold higher IC50 (about 65 μg/ml) than LoVo-parental cells was obtained (FIG. 2A). As shown in FIGS. 2B and 2C, oxaliplatin decreased the viability of both LoVo-parental and LoVo-OXAR cells in a dose-dependent manner. The LoVo-OXAR cells were significantly more resistant to oxaliplatin treatment and were morphologically different from LoVo-parental cells under 200× magnification.

### 2.3 Effects of the Compound (II) on the Viability of Colorectal Cancer Cells

LoVo-parental cells or LoVo-OXAR cells were seeded in 96-well plates at a density of 1×10⁴ cells/well (in triplicate) and cultured for 24 hours. The cells were then treated with the compound (II) at various concentrations (0, 125, 250, 500, 1000, 2000, or 4000 μg/ml) for 24 hours. After the medium was discarded, 100 μl of MTT solution (5.0 mg/ml) was added to each well and incubated for 4 to 5 hours at 37° C. until purple precipitates formed. The supernatant was removed, and dimethyl sulfoxide (DMSO) was added to each well to dissolve the blue formazan crystals. Absorbance at 570 nm (OD570) was measured using an ELISA plate reader (Molecular Devices, Palo Alto, CA, USA) to determine cell viability and the IC50 of the compound (II). Cell viability (percentage) was determined as follows: (OD570 of each test group/OD570 of the control group)×100%. An average of cell viability was calculated from triplicate experiments. IC50 was defined as the concentration of a test compound at which 50% of cells in a cell population die (i.e., a reduction of cell viability to 50%).

As shown in FIGS. 2D and 2E, the compound (II) reduced the viability of both colorectal cancer cells in a dose-dependent manner, and the IC50 values against LoVo-parental cells and LoVo-OXAR cells were about 500 μg/ml and about 2000 μg/ml, respectively. The results indicate the potency of the coordination compound to inhibit the survival of cancer cells, including chemo-resistant cancer cells.

2.4 Synergistic Effects of the Compound (II) in Combination with Other Chemotherapeutic Agents

To further investigate the influence of the compound (II) on the efficacy of chemotherapeutic agents, LoVo-parental cells or LoVo-OXAR cells were treated with the compound (II) (500 μg/ml for LoVo-parental cells; 2000 μg/ml for LoVo-OXAR cells), oxaliplatin (OXA, 20 μg/ml), irinotecan (20 μg/ml), 5-fluorouracil (5-FU, 20 μg/ml), or the combinations thereof for 24 hours, followed by determination of cell viability using MTT assay. As shown in FIG. 2F, commonly used chemotherapeutic agents, combined with the compound (II), were significantly more effective in inhibiting the survival of LoVo-parental and LoVo-OXAR cells than their application alone. The results indicate that the coordination compound disclosed herein can combat cancers alone or in combination with other chemotherapies.

### Example 3: Cytotoxic Effects of the Coordination Compound of Formula (II) on Lung Cancer Cells

### 3.1 Cell Culture

Human lung adenocarcinoma CL1 cells (Creative Biolabs, Shirley, NY, USA; hereinafter referred to as CL1-parental cells) and chemo-resistant subclones thereof were cultured in DMEM (Gibco™) supplemented with 10% FBS. BEAS-2B cells (95102433, Sigma-Aldrich) were cultured in LHC-9 medium (Gibco™, Thermo Fisher Scientific, Waltham, MA, USA). All cell cultures were maintained at 37° C. with 5% CO₂ supply.

### 3.2 Effects of the Compound (II) on the Viability of Lung Cancer Cells

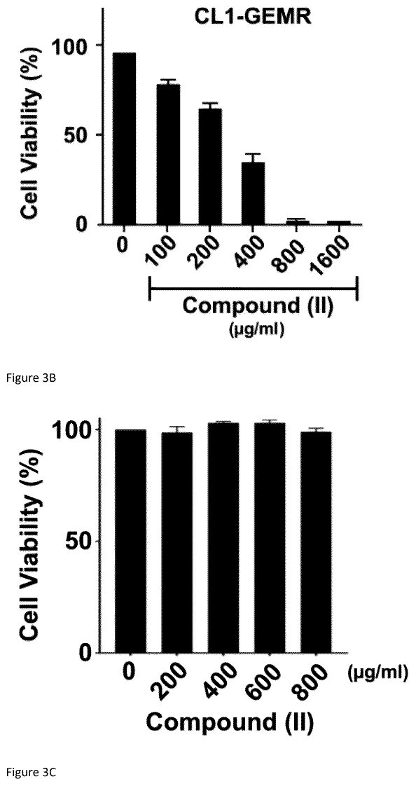

CL1-parental cells and gemcitabine-resistant CL1 cells were treated with the compound (II) at various concentrations (0, 100, 200, 400, 800, or 1600 μg/ml) for 24 hours, and MTT assay was used to determine the cytotoxic activity of the compound. As shown in FIGS. 3A and 3B, the compound (II) reduced the viability of both lung cancer cells in a dose-dependent manner.

### 3.3 Effects of the Compound (II) on the Viability of Non-Tumorigenic Lung Cells

The cytotoxicity of the compound (II) in non-cancerous cells was investigated using non-tumorigenic human bronchial epithelial BEAS-2B cells as a cell model. BEAS-2B cells were treated with varying doses (0, 200, 400, 600, or 800 μg/ml) of the compound (II) for 24 hours, followed by determination of cell viability using MTT assay. As shown in FIG. 3C, the compound (II) did not significantly inhibit the survival of BEAS-2B cells, demonstrating that the coordination compound disclosed herein does not impede the growth of non-cancerous human cells.

### Example 4: Induction of ER Stress-Mediated Apoptosis in Colorectal Cancer Cells by the Coordination Compound of Formula (II)

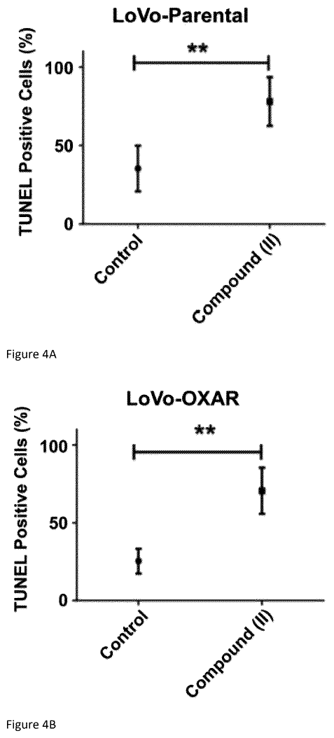

To evaluate whether the compound (II) induces apoptosis of cancer cells, terminal deoxynucleotidyl transferase dUTP nick-end labeling (TUNEL) assay was employed to detect apoptosis. In brief, LoVo-parental cells and LoVo-OXAR cells were treated with 500 μg/ml and 2000 μg/ml of the compound (II), respectively, for 24 hours before they were subject to TUNEL assay (In Situ Cell Death Detection Kit; Roche Applied Science, Indianapolis, IN, USA) and nuclear counterstain with 0.1 mg/ml 4′,6-diamidino-2-phenylindole (DAPI, Sigma-Aldrich, St. Louis, MO, USA). Lovo-parental or LoVo-OXAR cells treated with 0 μg/ml of the compound (II) were set as control groups. TUNEL-positive cells were examined by Olympus CKX53 microscope (Olympus, Shinjuku-ku, Tokyo, Japan). As shown in FIGS. 4A and 4B, compared to the control groups, a significant increase in apoptotic cell death (determined by the enhanced green fluorescence of apoptotic cells) was observed in both LoVo-parental cells and LoVo-OXAR cells when the compound (II) was applied.

Next, the proteins involved in the apoptosis induced by the compound (II) were further investigated. LoVo-parental cells and LoVo-OXAR cells were treated with the compound (II) (500 μg/ml for LoVo-parental cells; 2000 μg/ml for LoVo-OXAR cells), oxaliplatin (25 μg/ml for LoVo-parental cells; 45 μg/ml for LoVo-OXAR cells), or the combination thereof for 24 hours before the cells were harvested and lysed for western blotting. Lovo-parental or LoVo-OXAR cells receiving no compound treatment were set as control groups. Western blot analysis was conducted using primary antibodies against protein kinase RNA-like endoplasmic reticulum kinase (PERK), phosphorylated PERK (p-PERK), alpha subunit of eukaryotic translation initiation factor 2 (eIF2α), phosphorylated eIF2α (p-eIF2α), activating transcription factor 4 (ATF4), proliferating cell nuclear antigen (PCNA), glyceraldehyde 3-phosphate dehydrogenase (GAPDH), and caspase 3 (San-ta Cruz Biotechnology Inc. Santa Cruz, California, USA). GAPDH was used as an internal control for the normalization of protein expression. The results showed that eIF2α levels in LoVo-OXAR cells decreased while phosphorylation of eIF2α increased upon treatment of the compound (II) (FIG. 4C). Phosphorylated eIF2α has been known to inhibit the synthesis of various proteins participating in tumorigenesis. The results also demonstrated that the compound (II) elicits ER stress, enhancing the expression of ER stress markers, including PERK, p-eIF2α, and ATF4, and further activates caspase-3 cleavage, ultimately leading to apoptosis (FIG. 4C). Accordingly, the coordination compound disclosed herein can suppress cancer progression by acting as an inducer of ER stress aggravation and apoptosis in cancer cells.

### Example 5: Inhibitory Effects of the Coordination Compound of Formula (II) on Migration and Invasion of Colorectal Cancer Cells

### 5.1 Inhibition of Cancer Cell Migration and Invasion

Cancer cell migration and invasion are critical activities in tumor progression and metastasis. To evaluate whether the coordination compound has an impact on these activities, Transwell migration assay and invasion assay were performed on LoVo-parental and LoVo-OXAR cells. For each experiment, 5×10⁴ cells were resuspended in 200 μl serum-free media and loaded into the upper compartment (i.e., a 24-well insert with a porous membrane; pore size: 8 μm) of a Transwell chamber (Corning, New York, USA). The lower chamber was filled with DMEM containing 10% FBS as a chemoattractant. The cells were then incubated in the presence of the compound (II) (500 μg/ml for LoVo-parental cells; 2000 μg/ml for LoVo-OXAR cells), oxaliplatin (50 μg/ml), or the combination thereof. Lovo-parental or LoVo-OXAR cells receiving no compound treatment were set as control groups. In the migration assay, the incubation time was 48 hours. For the invasion assay, the insert was pre-coated with an extracellular matrix gel (BD Biosciences, Sparks, MD, USA), and the cells were incubated for 72 hours. At the end of each experiment, the cells on the upper surface of the membrane were removed while the cells on the lower surface were fixed and stained with Giemsa. The stained cells in five randomly selected areas were counted under a microscope (200-fold magnification). Invasion or migration rate was calculated as follows: Invasion (or migration) rate (%)=(average number of transmembrane cells in each test group/average number of transmembrane cells in the control group)×100%. An average invasion or migration rate was calculated from triplicate experiments.

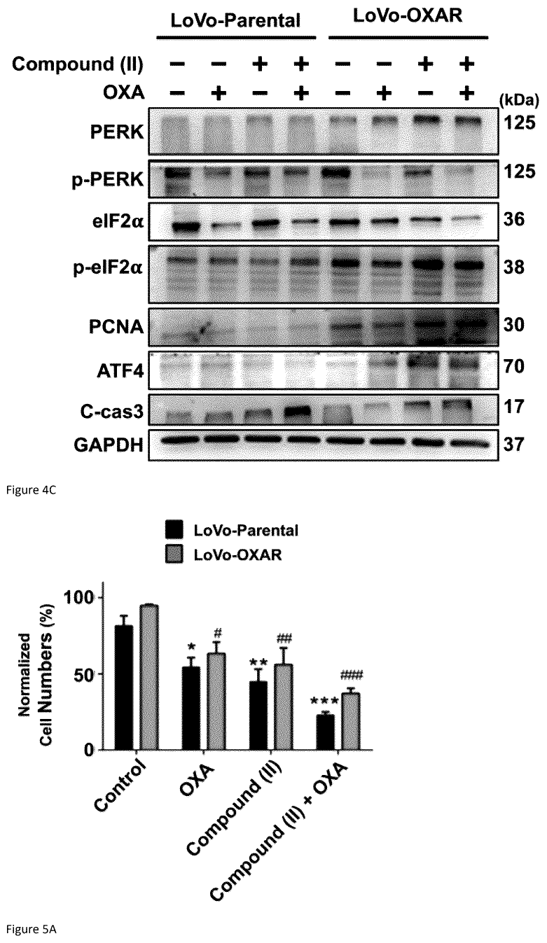

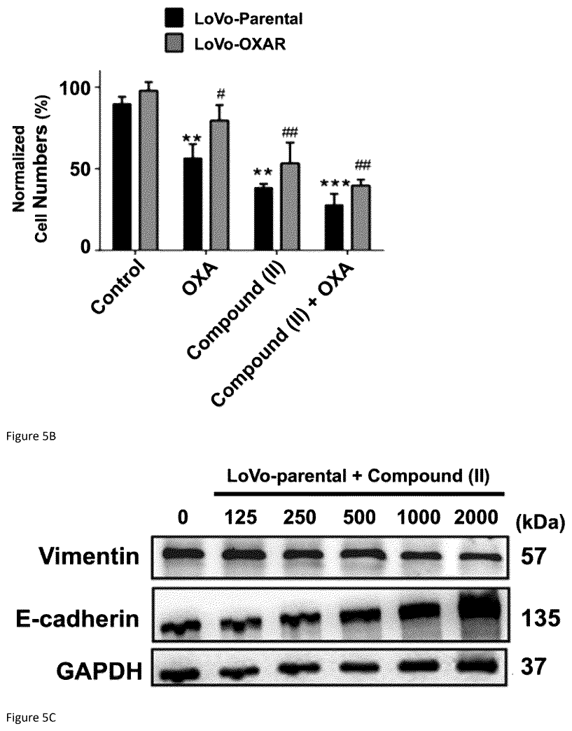

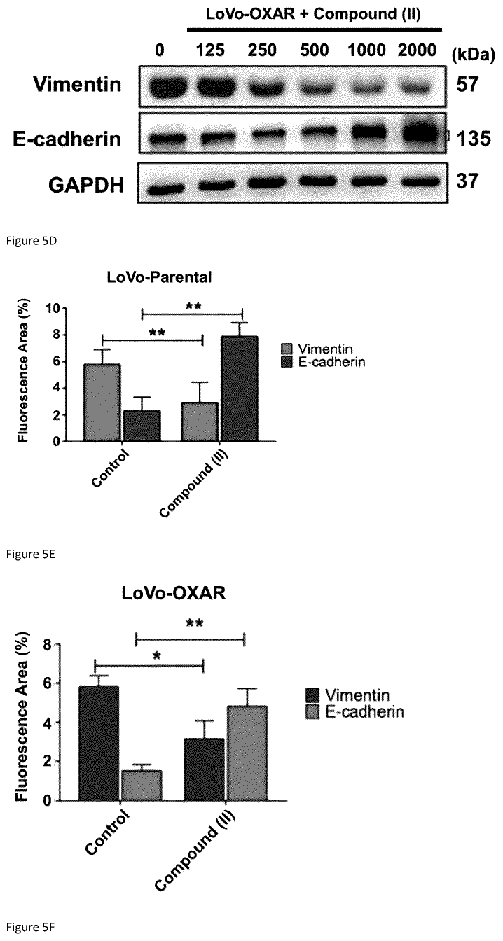

As shown in FIGS. 5A and 5B, either the compound (II) or oxaliplatin significantly decreased the migration and invasion rates of both LoVo-parental and LoVo-OXAR cells. In addition, the compound (II) combined with oxaliplatin synergistically reduced the migration and invasion rates of both cells to less than 50%. All results suggested that the coordination compound greatly inhibited the abilities of cancer cells, including chemo-resistant cancer cells, to migrate and invade.

### 5.2 Suppression of Epithelial-Mesenchymal Transition (EMT)

Vimentin and E-cadherin are molecular markers linked to tumor progression through their roles in EMT, which is a pathological process contributing to migration and invasion of cancer cells and tumor progression. Vimentin is a key component of intermediate filaments, which helps maintain cellular integrity and resistance to stress. E-cadherin plays a role in maintaining epithelial phenotypes by mediating contact inhibition when cells proliferate and reach confluence. To further investigate the influence of the compound (II) on EMT marker expression, western blotting, immunofluorescence detection, and quantitative real-time reverse transcription polymerase chain reaction (qRT-PCR) were performed in LoVo-parental and LoVo-OXAR cells treated with various doses of the compound (II) for 24 hours. Lovo-parental or LoVo-OXAR cells receiving no compound treatment were set as control groups.

### 5.2.1 Western Blotting

The cells after the indicated treatments were harvested and subject to western blot analysis using primary antibodies against vimentin, E-cadherin, and GAPDH (San-ta Cruz Biotechnology Inc. Santa Cruz, California, USA). GAPDH was used as an internal control. The results revealed that the protein expression of vimentin significantly reduced compared to the control groups while that of E-cadherin (also known as a tumor suppressor due to its role in contact inhibition) increased after LoVo-parental and LoVo-OXAR cells were treated with the compound (II) (FIGS. 5C and 5D).

### 5.2.2 Immunofluorescence Detection

LoVo-parental or LoVo-OXAR cells were seeded in 8-well plates at a density of 1×10⁴ cells/well and cultured in DMEM containing 10% FBS in the presence or absence of the compound (II) before being fixed with 4% paraformaldehyde in phosphate buffered saline (PBS). The cells were then permeabilized with 0.1% Triton X-100 in 0.1% sodium citrate and the well was blocked with blocking buffer (2% bovine serum albumin), followed by immunofluorescence staining. The cells were incubated with primary antibodies against vimentin and E-cadherin for 24 hours at 4° C. and subsequently incubated with fluorescent secondary antibodies for 1 hour at 25° C. before DAPI staining and observation using fluorescence microscopy. As shown in FIGS. 5E and 5F, treatment with the compound (II) significantly suppressed vimentin expression, whereas the expression of E-cadherin significantly increased.

5.2.3 qRT-PCR

Total RNA from the cells after the indicated treatments was extracted using the GeneJET RNA purification kit (ThermoFisher Scientific, Lithuania) and reverse transcribed to cDNA using a GScript First-Strand Synthesis Kit, a cDNA synthesis kit, and Oligo-dT primers according to the manufacturer's instructions. RT-PCR was performed using ORA^{T} SEE qPCR Green ROX L Mix, 2× (HighQu, USA). Primers for EMT markers (Vimentin, E-cadherin, TJP1, and FN1) and GAPDH (as endogenous control) were synthesized by Bio-ProTech based the sequences in TABLE 1. The 2^{-ΔΔCt} method was used to calculate a relative fold change in gene expression. The results showed that in both LoVo-parental and LoVo-OXAR cells, E-cadherin and TJP1 mRNA expression increased after treatment with the compound (II), whereas vimentin and FN1 mRNA expression decreased (FIGS. 5G and 5H). TJP1 is a marker of epithelial cells that disappears during EMT and cancer development. In contrast, FN1 is involved in the occurrence and development of various tumors and acts as a critical gene in gastric cancer. All data presented above suggest that the coordination compound can help inhibit cancer cell invasion and thus may be used as a complementary therapy to the current chemotherapies.

TABLE 1

| EMT | | SEQ |
|---|---|---|
| | | ID |
| markers | Primer sequence | NO |
| E- | Forward: 5′-CCCGGGACA | 1 |
| cadherin | ACGTTTATTAC-3′ | |
| | Reverse: 5′-GCTGGCTCA | 2 |
| | AGTCAAAGTCC-3′ | |
| Vimentin | Forward: 5′-TCCAGCAGCT | 3 |
| | TCCTGTAGGT-3′ | |
| | Reverse: 5′-GAGAACTTTG | 4 |
| | CCGTTGAAGC-3′ | |
| TJP1 | Forward: 5′-CCAGCTGGTA | 5 |
| | TGGGTTTCC-3′ | |
| | Reverse: 5′-TCTACTGTCC | 6 |
| | GTGCTATACATTGAGT-3′ | |
| FN1 | Forward: 5′-GACGCATCACT | 7 |
| | TGCACTTCT-3′ | |
| | Reverse: 5′-GCAGGTTTCCT | 8 |
| | CGATTATCCT-3′ | |
| GAPDH | Forward: 5′-GCACCGTCAA | 9 |
| | GGCTGAGAAC-3′ | |
| | Reverse: 5′-ATGGTGGTGA | 10 |
| | AGACGCCAGT-3′ | |

### Example 6: Treatment of Colorectal Cancers in Mice with the Coordination Compound of Formula (II)

### 6.1 Inhibition of Tumor Growth in Mice

Six-week-old male NU/NU mice were purchased from Bio LASCO Taiwan Co., Ltd (Taipei, Taiwan) and randomly assigned into four groups (Groups 1 to 4), each containing three mice. LoVo-parental or LoVo-OXAR cells (1×10⁶ cells in 100 μl DMEM) were subcutaneously injected into the mouse legs (day 0), and the mice were orally administered with the compound (II) or PBS every three days from day 1 after tumor cell inoculation (TABLE 2). Tumor volume and mice body weight were measured every three days. Tumor volume was measured with a caliper and calculated using the formula [(L×W×W)/2], where L and W represent tumor length and width, respectively. All mice were sacrificed on day 21, and the tumors were excised and weighed.

TABLE 2

| Tumor xenograft | Group | Treatment regime |
|---|---|---|
| LoVo-OXAR cells | 1 (Control) | PBS |
| | 2 | Compound (II) in PBS, 100 mg/kg, |
| | | every three days |
| LoVo-parental cells | 3 (Control) | PBS |
| | 4 | Compound (II) in PBS, 100 mg/kg, |
| | | every three days |

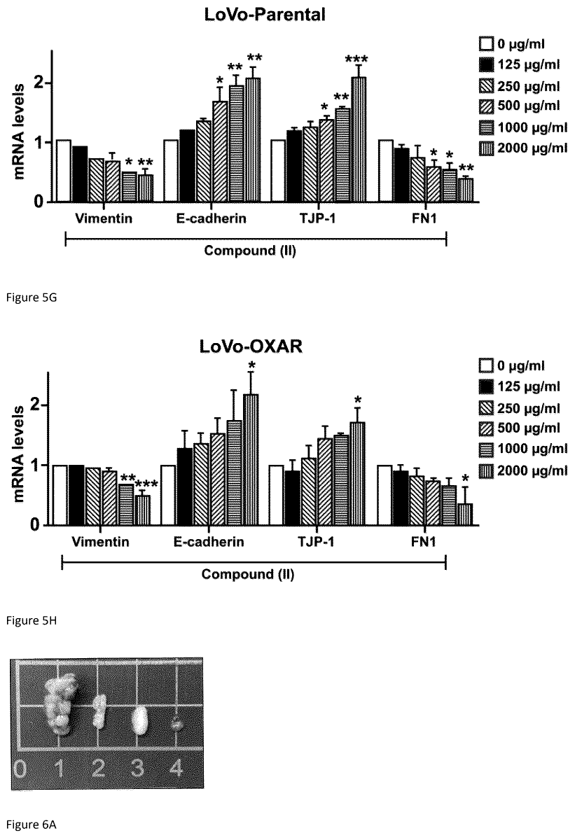

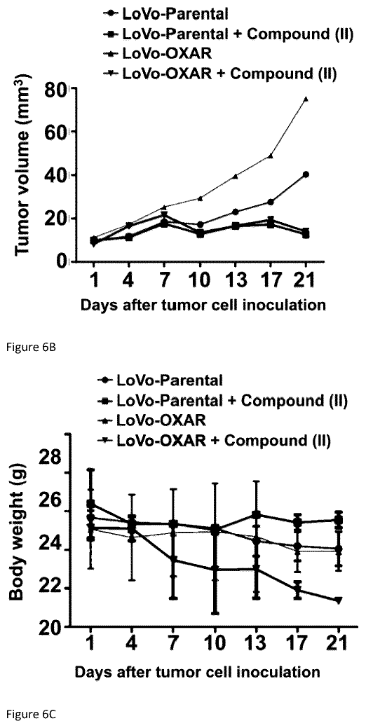

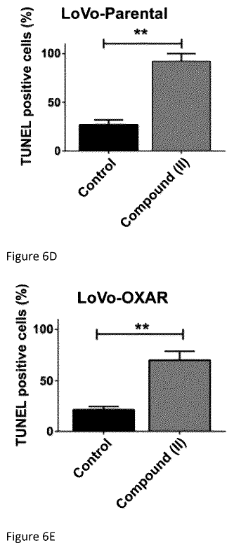

As shown in FIGS. 6A and 6B, treatment with the compound (II) for about three weeks significantly reduced the tumor growth in mice injected with either LoVo-OXAR cells or LoVo-parental cells, demonstrating that the coordination compound disclosed herein may treat cancers, including chemo-resistant cancers, in a subject. In addition, the body weight of the mice remained constant except in the groups inoculated with chemo-resistant tumor cells; the body weight loss was probably due to the aggressiveness of the chemo-resistant tumor (FIG. 6C). However, all mice were healthy after treatment with the compound (II).

Moreover, the LoVo-parental and LoVo-OXAR tumor tissues were collected and subject to TUNEL assay. The results indicated that administering the compound (II) to the mice induced significantly higher apoptosis in both LoVo-parental and LoVo-OXAR tumors (FIGS. 6D and 6E).

### 6.2 Inhibition of Metastasis in Mice

Six-week-old male NU/NU mice were purchased from Bio LASCO Taiwan Co., Ltd (Taipei, Taiwan) and randomly assigned into six groups (Groups 1 to 6), each containing three mice. LoVo-parental or LoVo-OXAR cells (1×10⁶ cells in 100 μl DMEM) were injected into the mouse tail vein (day 0), and the mice were orally administered with the compound (II) or PBS five days per week for three weeks from day 1 after the tumor cell inoculation (TABLE 3). All mice were sacrificed on day 30, and the mouse lung, kidney, spleen, and heart were examined for metastatic cancer cells.

TABLE 3

| Tumor xenograft | Group | Treatment regime |
|---|---|---|
| LoVo-parental cells | 1 (Control) | PBS |
| | 2 (Low dose) | Compound (II) in PBS, |
| | | 10 mg/kg/day |
| | 3 (High dose) | Compound (II) in PBS, |
| | | 100 mg/kg/day |
| LoVo-OXAR cells | 4 (Control) | PBS |
| | 5 (Low dose) | Compound (II) in PBS, |
| | | 10 mg/kg/day |
| | 6 (High dose) | Compound (II) in PBS, |
| | | 100 mg/kg/day |

As shown in FIGS. 6F and 6G, treatment with a high dose of the compound (II) effectively inhibited the metastasis of LoVo-parental or LoVo-OXAR cells to the lung, kidney, spleen, and heart, compared to the low-dose treatment. The results revealed the potential of the coordination compound to impede tumor development and metastasis.

### Example 7: Cytotoxic Effects of the Coordination Compound of Formula (II) on Liver Cancer Cells

### 7.1 Cell Culture

Human hepatocellular carcinoma HA22T cells (commercially available from BCRC under the number BCRC 60168; hereinafter referred to as HA22T-parental cells) and chemo-resistant subclones thereof were cultured in DMEM supplemented with 10% FBS. HA22T cells resistant to SAHA (an HDAC inhibitor) was established according to a selection process similar to that described in Example 2.2. All cell cultures were maintained at 37° C. with 5% CO₂ supply.

### 7.2 Effects of the Compound (II) on the Viability of Liver Cancer Cells

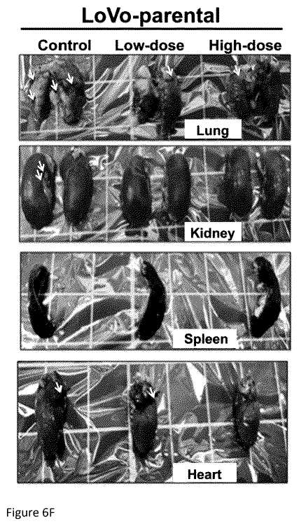

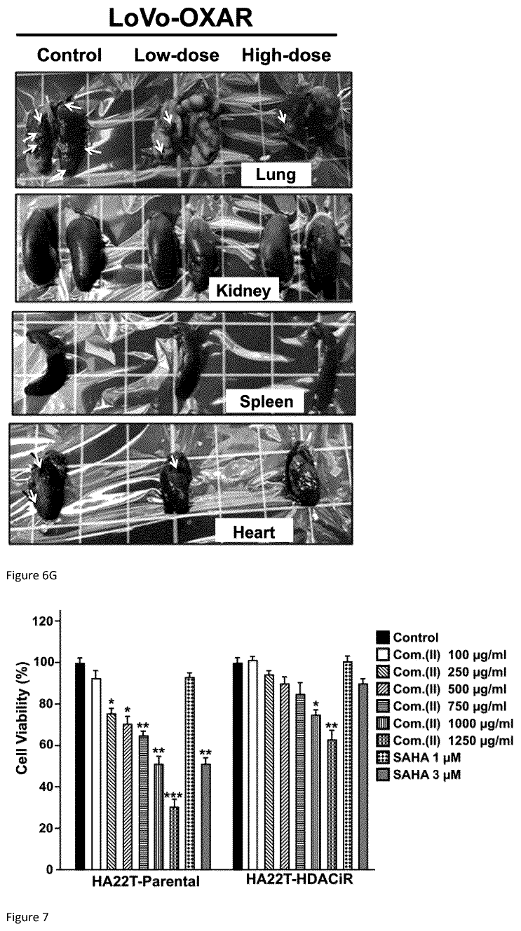

HA22T-parental cells and SAHA-resistant HA22T cells (referred to as HA22T-HDACiR cells) were treated with the compound (II) at various concentrations (0, 100, 250, 500, 750, 1000, or 1250 μg/ml) or SAHA (1 or 3 μM) for 24 hours, and MTT assay was used to determine the cytotoxic activity of the compound. As shown in FIG. 7, the compound (II) reduced the viability of both liver cancer cells in a dose-dependent manner, suggesting the potential of the compound (II) to treat liver cancer. The IC50 value against HA22T-parental cells was approximately 1000 μg/ml.

### Example 8: Cytotoxic Effects of the Coordination Compound of Formula (II) on Prostate Cancer Cells

### 8.1 Cell Culture

Human prostate carcinoma LNCaP cells (commercially available from ATCC under the number CRL-1740) were cultured in RPMI-1640 medium (Gibco™, Thermo Fisher Scientific, Waltham, MA, USA) supplemented with 10% FBS. The cell culture was maintained at 37° C. with 5% CO₂ supply.

### 8.2 Effects of the Compound (II) on the Viability of Prostate Cancer Cells

LNCaP cells were treated with the compound (II) at various concentrations (0, 100, 200, 400, 800, or 1600 μg/ml) for 24 hours, and MTT assay was used to determine the cytotoxic activity of the compound. As shown in FIG. 8, the compound (II) reduced the viability of the prostate cancer cells in a dose-dependent manner, suggesting the potential of the compound (II) to treat prostate cancer. The IC50 value against LNCaP cells was approximately 1033 μg/ml.

### Example 9: Cytotoxic and Migration-Inhibitory Effects of the Coordination Compound of Formula (II) on Osteosarcoma Cells

### 9.1 Cell Culture

Human osteosarcoma 143B cells (commercially available from ATCC under the number CRL-8303) were cultured in DMEM supplemented with 10% FBS and 1% sodium pyruvate. The cell culture was maintained at 37° C. with 5% CO₂ supply.

### 9.2 Effects of the Compound (II) on the Viability of Osteosarcoma Cells

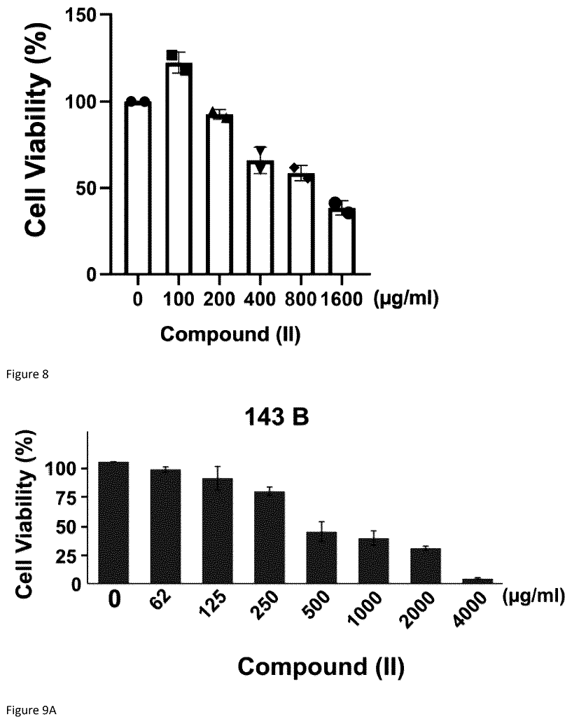

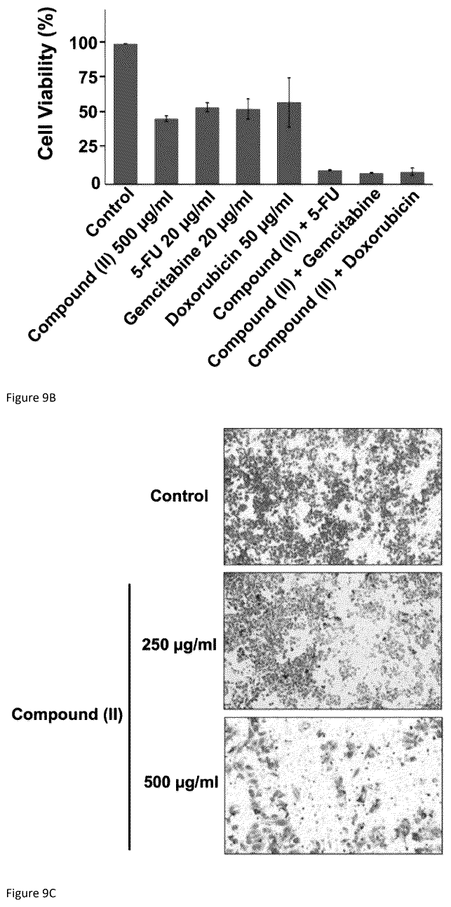

143B cells were treated with the compound (II) at various concentrations (0, 62, 125, 250, 500, 1000, 2000, or 4000 μg/ml) for 24 hours, and MTT assay was used to determine the cytotoxic activity of the compound. As shown in FIG. 9A, the compound (II) reduced the viability of the osteosarcoma cells in a dose-dependent manner, suggesting the potential of the compound (II) to treat bone cancer. The IC50 value against 143B cells was approximately 500 μg/ml. Furthermore, the compound (II) (500 μg/ml) combined with 5-fluorouracil (20 μg/ml), gemcitabine (20 μg/ml), or doxorubicin (50 μg/ml) inhibited 143B cell survival significantly greater than using either one chemotherapeutic agent alone (FIG. 9B), indicating that the coordination compound may serve as a complementary anticancer drug to conventional chemotherapeutic agents.

### 9.3 Inhibition of the Crawling and Epithelial-Mesenchymal Transition of Osteosarcoma Cells

Transwell migration assay, as described in Example 5.1, was performed on 143B cells to investigate how the compound (II) would affect the migration ability of bone cancer cells. As shown in FIG. 9C, the crawling of 143B cells was inhibited by the compound (II) in a dose-dependent manner. In addition, western blot analysis showed that treatment with the compound (II) significantly decreased vimentin expression while increasing E-cadherin expression (FIG. 9D), suggesting the inhibitory potential of the coordination compound on the EMT process and development of invasive cancer cells.

### Example 10: Cytotoxic Effects of the Coordination Compound of Formula (II) on Glioblastoma Cells

### 10.1 Cell Culture

Human brain glioblastoma multiforme GBM 8401 cells (commercially available from BCRC under the number BCRC 60163) were cultured in RPMI 1640 medium supplemented with 10% FBS. The cell culture was maintained at 37° C. with 5% CO₂ supply.

### 10.2 Effects of the Compound (II) on the Viability of Glioblastoma Cells

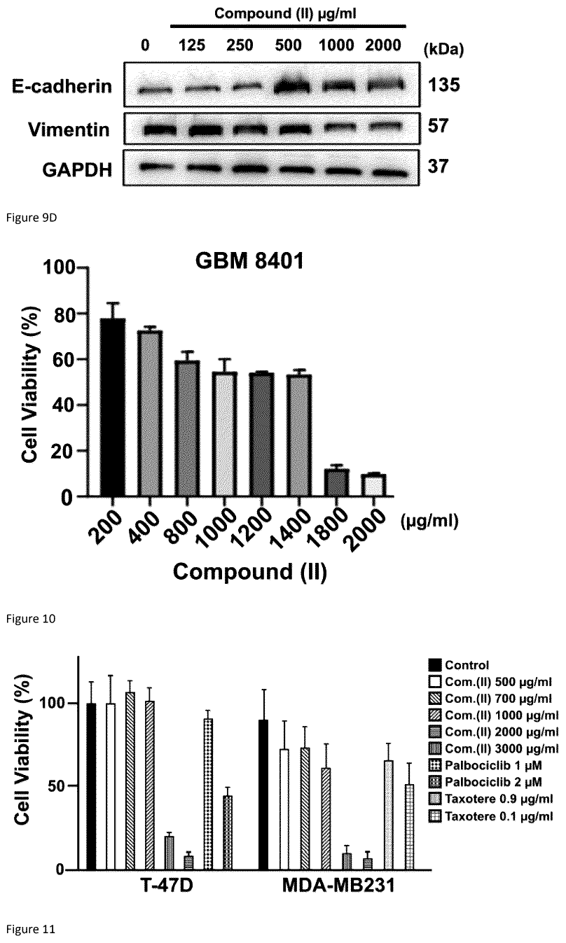

GBM 8401 cells were treated with the compound (II) at various concentrations (0, 200, 400, 800, 1000, 1200, 1400, or 1800 μg/ml) for 24 hours, and MTT assay was used to determine the cytotoxic activity of the compound. As shown in FIG. 10, the compound (II) reduced the viability of the glioblastoma cells in a dose-dependent manner, suggesting the potential of the compound (II) to treat brain cancer.

### Example 11: Cytotoxic Effects of the Coordination Compound of Formula (II) on Breast Cancer Cells

### 11.1 Cell Culture

Human breast cancer cells T-47D (commercially available from ATCC under the number HTB-133) and MDA-MB231 (commercially available from ATCC under the number HTB-26) were cultured in RPMI 1640 medium supplemented with 10% FBS. The cell culture was maintained at 37° C. with 5% CO₂ supply.

### 11.2 Effects of the Compound (II) on the Viability of Breast Cancer Cells

T-47D and MDA-MB231 cells were treated with the compound (II) at various concentrations (0, 500, 700, 1000, 2000, or 3000 μg/ml) for 24 hours, and MTT assay was used to determine the cytotoxic activity of the compound. The results showed that the compound (II) reduced the viability of both breast cancer cells in a dose-dependent manner, and its cell-killing effect was more prominent in MDA-MB231 cells (FIG. 11), a highly invasive triple-negative breast cancer cell line characterized by EMT and resistance to doxorubicin.

### Example 12: Cytotoxic Effects of the Coordination Compound of Formula (II) on Bladder Cancer Cells

### 12.1 Cell Culture

Human bladder carcinoma T24 cells (commercially available from ATCC under the number HTB-4) were cultured in RPMI 1640 medium supplemented with 10% FBS. The cell culture was maintained at 37° C. with 5% CO₂ supply.

### 12.2 Effects of the Compound (II) on the Viability of Bladder Cancer Cells

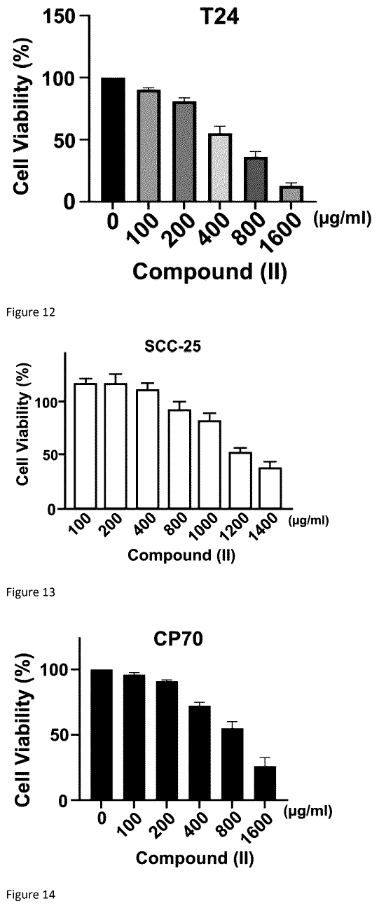

T24 cells were treated with the compound (II) at various concentrations (0, 100, 200, 400, 800, or 1600 μg/ml) for 24 hours, and MTT assay was used to determine the cytotoxic activity of the compound. As shown in FIG. 12, the compound (II) reduced the viability of the bladder carcinoma cells in a dose-dependent manner, suggesting the potential of the compound (II) to treat bladder cancer.

### Example 13: Cytotoxic Effects of the Coordination Compound of Formula (II) on Oral Cancer Cells

### 13.1 Cell Culture

Human oral squamous cell carcinoma (OSCC)SCC-25 cells (commercially available from ATCC under the number CRL-1628) were cultured in DMEM/F12 supplemented with 10% FBS. The cell culture was maintained at 37° C. with 5% CO₂ supply.

### 13.2 Effects of the Compound (II) on the Viability of Oral Cancer Cells

SCC-25 cells were treated with the compound (II) at various concentrations (0, 100, 200, 400, 800, 1000, 1200, or 1400 μg/ml) for 24 hours, and MTT assay was used to determine the cytotoxic activity of the compound. As shown in FIG. 13, the compound (II) reduced the viability of the OSCC cells in a dose-dependent manner, suggesting the potential of the compound (II) to treat oral cancer.

### Example 14: Cytotoxic Effects of the Coordination Compound of Formula (II) on Ovarian Cancer Cells

### 14.1 Cell Culture

Human ovarian endometrioid caner CP70 cells (a cisplatin-resistant cell line obtained from Dr. Chih-Yang Huang at Hualien Tzu Chi Hospital (Taiwan) and established from the ovarian cancer cell line A2780, which was commercially available from the European Collection of Authenticated Cell Cultures (ECACC) under the number 93112519) were cultured in RPMI 1640 medium supplemented with 10% FBS. The cell culture was maintained at 37° C. with 5% CO₂ supply.

### 14.2 Effects of the Compound (II) on the Viability of Ovarian Cancer Cells

CP70 cells were treated with the compound (II) at various concentrations (0, 100, 200, 400, 800, or 1600 μg/ml) for 24 hours, and MTT assay was used to determine the cytotoxic activity of the compound. As shown in FIG. 14, the compound (II) reduced the viability of the ovarian cancer cells in a dose-dependent manner, suggesting the potential of the compound (II) to treat ovarian cancer.

### Example 15: Cytotoxic Effects of the Coordination Compound of Formula (II) on Leukemia Cells

### 15.1 Cell Culture

Human T-acute lymphoblastic leukemia Jurkat cells (resistant to cisplatin and commercially available from ATCC under the number TIB-512) were cultured in RPMI 1640 medium supplemented with 10% FBS. The cell culture was maintained at 37° C. with 5% CO₂ supply.

### 15.2 Effects of the Compound (II) on the Viability of Leukemia Cells

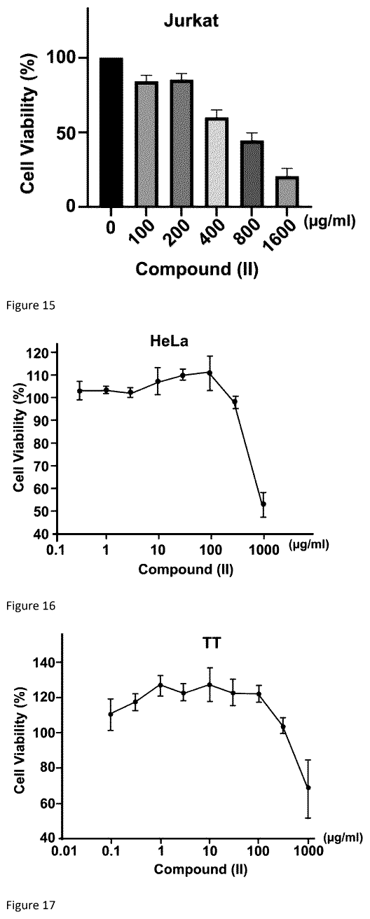

Jurkat cells were treated with the compound (II) at various concentrations (0, 100, 200, 400, 800, or 1600 μg/ml) for 24 hours, and MTT assay was used to determine the cytotoxic activity of the compound. As shown in FIG. 15, the compound (II) reduced the viability of the Jurkat cells in a dose-dependent manner, suggesting the potential of the compound (II) to treat leukemia.

### Example 16: Cytotoxic Effects of the Coordination Compound of Formula (II) on Cervical Cancer Cells

### 16.1 Cell Culture

Human cervical carcinoma HeLa cells (commercially available from ATCC under the number CCL-2) were cultured in DMEM supplemented with 10% FBS. The cell culture was maintained at 37° C. with 5% CO₂ supply.

### 16.2 Effects of the Compound (II) on the Viability of Cervical Cancer Cells

HeLa cells were treated with the compound (II) at various concentrations (0.5, 1, 5, 10, 50, 100, 500, or 1000 μg/ml) for 48 hours, and MTT assay was used to determine the cytotoxic activity of the compound. As shown in FIG. 16, the compound (II) reduced the viability of the metastatic Hela cells in a dose-dependent manner, suggesting the potential of the compound (II) to treat cervical cancer, including metastatic cervical cancer.

### Example 17: Cytotoxic Effects of the Coordination Compound of Formula (II) on Thyroid Cancer Cells

### 17.1 Cell Culture

Human medullary thyroid carcinoma TT cells (commercially available from ATCC under the number CRL-1803) were cultured in F12K medium (Gibco™, Thermo Fisher Scientific, Waltham, MA, USA) supplemented with 10% FBS. The cell culture was maintained at 37° C. with 5% CO₂ supply.

### 17.2 Effects of the Compound (II) on the Viability of Thyroid Cancer Cells

TT cells were treated with the compound (II) at various concentrations (0.1, 0.5, 1, 5, 10, 50, 100, 500, or 1000 μg/ml) for 72 hours, and MTT assay was used to determine the cytotoxic activity of the compound. As shown in FIG. 17, the compound (II) reduced the viability of the TT cells in a dose-dependent manner, suggesting the potential of the compound (II) to treat thyroid cancer.

### Example 18: Cytotoxic Effects of the Coordination Compound of Formula (II) on Skin Cancer Cells

### 18.1 Cell Culture

Human epidermoid carcinoma A431 cells (commercially available from ATCC under the number CRL-1555) were cultured in DMEM supplemented with 10% FBS. The cell culture was maintained at 37° C. with 5% CO₂ supply.

### 18.2 Effects of the Compound (II) on the Viability of Skin Cancer Cells

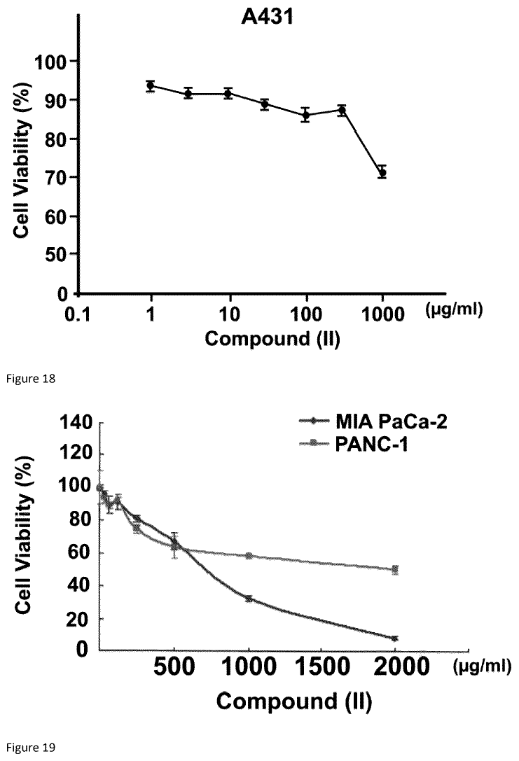

A431 cells were treated with the compound (II) at various concentrations (1, 5, 10, 50, 100, 500, or 1000 μg/ml) for 48 hours, and MTT assay was used to determine the cytotoxic activity of the compound. As shown in FIG. 18, the compound (II) reduced the viability of the A431 cells in a dose-dependent manner, suggesting the potential of the compound (II) to treat skin cancer.

### Example 19: Cytotoxic Effects of the Coordination Compound of Formula (II) on Pancreatic Cancer Cells

### 19.1 Cell Culture

Human pancreatic carcinoma MIA PaCa-2 cells (commercially available from ATCC under the number CRL-1420) were cultured in DMEM supplemented with 10% FBS, 1 mM sodium pyruvate, and 2.5% horse serum. Human pancreatic epithelioid carcinoma PANC-1 cells (commercially available from ATCC under the number CRL-1469) were cultured in DMEM supplemented with 10% FBS and 1 mM sodium pyruvate. All cell cultures were maintained at 37° C. with 5% CO₂ supply.

### 19.2 Effects of the Compound (II) on the Viability of Pancreatic Cancer Cells

MIA PaCa-2 and PANC-1 cells were treated with the compound (II) at various concentrations (0, 31, 62.5, 125, 250, 500, 1000, or 2000 μg/ml) for six days, and MTT assay was used to determine the cytotoxic activity of the compound. The results showed that the compound (II) reduced the viability of both pancreatic cancer cells in a dose-dependent manner (FIG. 19), suggesting the potential of the compound (II) to treat pancreatic cancer. The IC50 value against MIA PaCa-2 cells and PANC-1 cells were approximately 660 μg/ml and 1700 μg/ml, respectively.

### Example 20: Treatment of Pancreatic Cancers in Mice with the Coordination Compound of Formula (II)

Female C.B17/SCID mice were purchased from the National Laboratory Animal Center (Taipei, Taiwan) and randomly assigned into five groups (Groups 1 to 5), each containing seven mice. PANC-1 cells (1×10⁶ cells in 50 μl PBS), mixed with an equal volume of basement matrigel (BD Biosciences, Franklin Lakes, NJ, USA), were subcutaneously injected into the mouse legs (day 0). After tumors reached a volume ranging between 200 and 300 mm³ (day 12), the mice were orally administered with the compound (II) or PBS or intravenously injected with Eli Lilly's Gemzar (gemcitabine for injection) for 14 days (TABLE 4). Tumor volume was monitored for 14 more days before all mice were sacrificed. Tumor volume was measured with a caliper and calculated using the formula [(L×W×W)/2], where L and W represent tumor length and width, respectively. Tumor growth inhibition was determined as follows: inhibition (%)=[1−(Vt/Vc)]×100, where Vt refers to net tumor growth in mice under compound treatment and Vc refers to net tumor growth in control mice (no compound treatment). The net tumor growth was calculated by subtracting the tumor volume determined upon the first treatment from the tumor volume determined subsequently.

TABLE 4

| Tumor xenograft | Group | Treatment regime |
|---|---|---|
| PANC-1 cells | 1 (Control) | PBS |
| | 2 (Positive control) | Gemzar, 200 mg/kg, twice a week |
| | 3 | Compound (II) in PBS, |
| | | 100 mg/kg, once a day |
| | 4 | Compound (II) in PBS, |
| | | 150 mg/kg, once a day |
| | 5 | Compound (II) in PBS, |
| | | 150 mg/kg, twice a day |

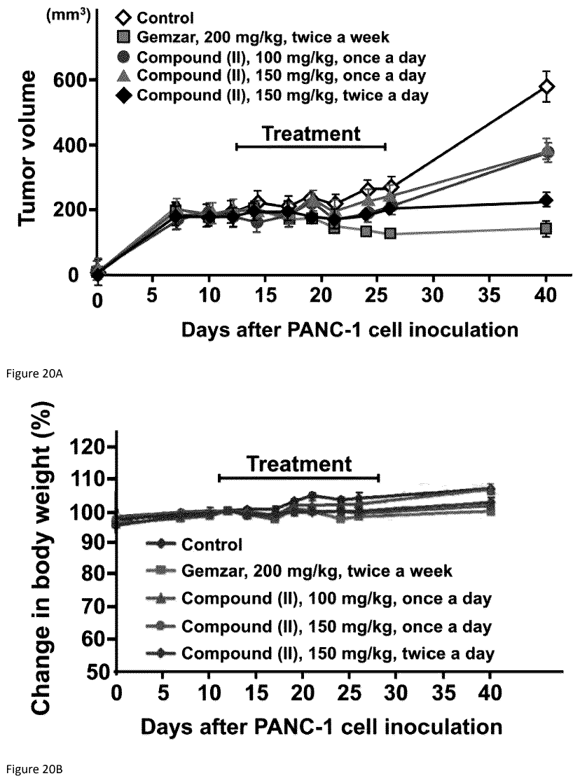

As shown in FIG. 20A, there was no significant change in tumor growth for each group during the 14-day treatment period. However, tumor growth inhibition (TGI) was observed 14 days after the treatment period. According to TABLE 5, treatment with the compound (II) resulted in about 51% to 88% inhibition in tumor growth depending on the dosing regime, and treatment with Gemzar (a chemotherapeutic agent commonly used to treat pancreatic cancer) caused about 112% inhibition. The data demonstrated that the compound (II) can treat pancreatic tumors. In addition, the body weight of the mice in Groups 3-5 remained constant (FIG. 20B), indicating that the compound (II) at a therapeutically effective dose is not toxic to mice.

TABLE 5

| Group | TGI (%) | Change in body weight |
|---|---|---|
| 1 | NA | 107 ± 4% |
| 2 | 112 ± 6% | 100 ± 4% |
| 3 | 51 ± 8% | 107 ± 5% |
| 4 | 51 ± 6% | 102 ± 3% |
| 5 | 88 ± 6% | 103 ± 4% |

In summary, the coordination compound disclosed herein effectively inhibits the survival of various cancer cells, including chemo-resistant cancer cells, thereby suppressing cancer cell proliferation and tumor growth. The coordination compound also inhibits the migration and invasion of cancer cells, which helps repress tumor progression and metastasis. Accordingly, the coordination compound may be utilized to manufacture a medicament for treating cancers, even metastatic cancers. Moreover, the synergistic effects of the coordination compound with current chemotherapeutic agents such as oxaliplatin suggest the compound may be used to formulate drug combinations to improve cancer treatment efficacy further.

1. A coordination compound represented by formula (I): [XZ₂(CH₃CO₂)₆(H₂O)₄(OH)₂]NO₃, wherein X is a trivalent ion of chromium (Cr) or molybdenum (Mo), and Z is a trivalent ion of iron (Fe), ruthenium (Ru), or osmium (Os).

2. A method for inhibiting survival or proliferation of a cancer cell, comprising contacting the cancer cell with an effective amount of the coordination compound according to claim 1.

3. The method of claim 2, wherein the coordination compound is represented by formula (11):

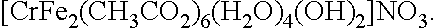

4. The method of claim 2, wherein the cancer is selected from colorectal cancer, lung cancer, liver cancer, pancreatic cancer, bone cancer, brain cancer, breast cancer, ovarian cancer, cervical cancer, prostate cancer, bladder cancer, blood cancer, gastric cancer, skin cancer, head-and-neck cancer, or thyroid cancer.

5. The method of any one of claims 2 to 4, wherein the cancer cell is invasive and/or resistant to a chemotherapeutic agent.

6. The method of any one of claims 2 to 5, further comprising contacting the cancer cell with a chemotherapeutic agent selected from oxaliplatin, irinotecan, 5-fluorouracil, gemcitabine, doxorubicin, or any combination thereof.

7. The method of any one of claims 2 to 6, wherein the coordination compound induces endoplasmic reticulum (ER) stress-mediated apoptosis of the cancer cell.

8. A method for inhibiting migration or invasion of a cancer cell, comprising contacting the cancer cell with an effective amount of the coordination compound according to claim 1.

9. The method of claim 8, wherein the coordination compound is represented by formula (11):

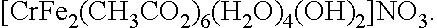

10. The method of claim 8, wherein the cancer is colorectal cancer or bone cancer.

11. The method of any one of claims 8 to 10, wherein the cancer cell is invasive and/or resistant to a chemotherapeutic agent.

12. The method of any one of claims 8 to 11, wherein the coordination compound suppresses epithelial-mesenchymal transition of the cancer cell.

13. A method for treating a cancerous tumor, comprising administering to a subject in need an effective amount of the coordination compound according to claim 1.

14. The method of claim 13, wherein the coordination compound is represented by formula (11):

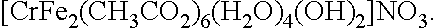

15. The method of claim 13, wherein the cancerous tumor is selected from colorectal cancer, lung cancer, liver cancer, pancreatic cancer, bone cancer, brain cancer, breast cancer, ovarian cancer, cervical cancer, prostate cancer, bladder cancer, blood cancer, gastric cancer, skin cancer, head-and-neck cancer, or thyroid cancer.

16. The method of any one of claims 13 to 15, wherein the cancerous tumor includes a cancer cell being invasive and/or resistant to a chemotherapeutic agent.

17. A pharmaceutical composition, comprising an effective amount of the coordination compound according to claim 1 and a pharmaceutically acceptable carrier.

18. The pharmaceutical composition of claim 17, wherein the coordination compound is represented by formula (II): [CrFe₂(CH₃CO₂)₆(H₂O)₄(OH)₂]NO₃.

19. The pharmaceutical composition of claim 17, further comprising an additional pharmaceutically active agent.

20. The pharmaceutical composition of claim 19, wherein the additional pharmaceutically active agent is a chemotherapeutic agent selected from oxaliplatin, irinotecan, 5-fluorouracil, gemcitabine, doxorubicin, or any combination thereof.


SEQUENCE LISTING (10 sequences)

SEQ ID NO: 1 - DNA, 20 bp, synthetic construct
  source 1..20: mol_type = other DNA
cccgggacaacgtttattac

SEQ ID NO: 2 - DNA, 20 bp, synthetic construct
  source 1..20: mol_type = other DNA
gctggctcaagtcaaagtcc

SEQ ID NO: 3 - DNA, 20 bp, synthetic construct
  source 1..20: mol_type = other DNA
tccagcagcttcctgtaggt

SEQ ID NO: 4 - DNA, 20 bp, synthetic construct
  source 1..20: mol_type = other DNA
gagaactttgccgttgaagc

SEQ ID NO: 5 - DNA, 19 bp, synthetic construct
  source 1..19: mol_type = other DNA
ccagctggtatgggtttcc

SEQ ID NO: 6 - DNA, 26 bp, synthetic construct
  source 1..26: mol_type = other DNA
tctactgtccgtgctatacattgagt

SEQ ID NO: 7 - DNA, 20 bp, synthetic construct
  source 1..20: mol_type = other DNA
gacgcatcacttgcacttct

SEQ ID NO: 8 - DNA, 21 bp, synthetic construct
  source 1..21: mol_type = other DNA
gcaggtttcctcgattatcct

SEQ ID NO: 9 - DNA, 20 bp, synthetic construct
  source 1..20: mol_type = other DNA
gcaccgtcaaggctgagaac

SEQ ID NO: 10 - DNA, 20 bp, synthetic construct
  source 1..20: mol_type = other DNA
atggtggtgaagacgccagt